(19) United States
(12) Patent Application Publication
(10) Pub. No.: US 2020/0386761 A1
(43) Pub. Date: Dec. 10, 2020

(54) SPECIFIC BIOMARKER SET FOR NON-INVASIVE DIAGNOSIS OF LIVER CANCER

(71) Applicant: Dragon Victory Development Ltd., Hong Kong (HK)

(72) Inventors: Cornelia Wing Yin MAN, Hong Kong (HK); Norman Fung Man WAI, Vancouver (CA); Bing Lou WONG, Irvine, CA (US); Benjamin Chi Yin WAI, Burnaby (CA)

(73) Assignee: Dragon Victory Development Ltd., Hong Kong (HK)

(21) Appl. No.: 16/825,515

(22) Filed: Mar. 20, 2020

Related U.S. Application Data
(63) Continuation-in-part of application No. 15/331,472, filed on Oct. 21, 2016, now Pat. No. 10,620,209.
Application No. 15/331,472 is a continuation of application No. 14/321,867, filed on Jul. 2, 2014, now Pat. No. 9,506,925.

(51) Int. Cl. G01N 33/574; G01N 33/564; G01N 21/64

(52) U.S. Cl. CPC G01N 33/57438; G01N 33/564; G01N 21/6428; G01N 2458/00; G01N 2021/6441; G01N 2333/9108; G01N 2333/475; G01N 2333/50; G01N 2333/54; G01N 2333/82; G01N 2333/90283; G01N 2333/4703

(57) ABSTRACT

Cells within liver tumor mass comprise a unique set of proteins/tumor antigens when compared to the normal liver tissues epithelial cells juxtaposed to the tumor. The presence of tumor antigens couples the production of auto-antibodies against these tumor antigens. The present invention relates to the identification and elucidation of a protein set that can act as a novel marker set for liver cancer diagnosis and prognosis. Specifically, it relates to a kit that enables diagnostic and prognostic measurement of auto-antibodies in serum of liver cancer patients. The present invention provides a non-invasive, specific, sensitive, and cost effective detection and quantification method by evaluating a set of validated liver cancer proteins/tumor antigens, which includes Bmi-1, VCC1, SUMO-4, RhoA, TXN, ET-1, UBE2C, HDGF2, FGF21, LECT2, SOD1, STMN4, Midkine, IL-17A, IL26, or DCP to complement the conventional diagnostic methods.

### RELATED APPLICATIONS

This is a continuation-in-part application of U.S. application Ser. No. 15/331,472, which was filed on Oct. 21, 2016, which is a continuation of U.S. application Ser. No. 14/321,867 filed on Jul. 2, 2014, which issued as U.S. Pat. No. 9,506,925, the contents of each incorporated by reference in their entirety.

### COPYRIGHT NOTICE/PERMISSION

A portion of the disclosure of this patent document contains material which is subject to copyright protection. The copyright owner has no objection to the facsimile reproduction by anyone of the patent document or the patent disclosure as it appears in the Patent and Trademark Office patent file or records, but otherwise reserves all copyright rights whatsoever. The following notice applies to the processes, experiments, and data as described below and in the drawings attached hereto: Copyright © 2014, Vision Global Holdings Limited, All Rights Reserved.

### TECHNICAL FIELD

The present invention provides a detection and quantification method for specific and novel Hepatocellular Carcinoma (HCC) tumor biomarkers, by measuring the corresponding auto-antibodies in liver cancer patients' sera. The set of HCC biomarkers includes Bmil, VCC1, SUMO-4, RhoA, TXN, ET-1, UBE2C, HDGF2 (which is also known as HDGFRP3), FGF21, LECT2, SOD1, STMN4, Midkine, IL-17A, IL26, and DCP. More specifically, this invention further provides a high throughput and sensitive test kit readily available to take patients' peripheral serum samples and detect liver cancers early and in a non-invasive manner by measuring the auto-antibodies against at least one of the HCC biomarkers selected from the above mentioned HCC biomarker set. The present invention further allows identification of signature HCC biomarker patterns for staging, as well as the detection of recurrences during a monitoring period of post-chemotherapeutic treatment. The present invention also supports automatic data analysis.

### BACKGROUND OF INVENTION

Hepatocellular carcinoma (HCC) is the second most prevalent cancer in China, which covers 5.7% of the total population. See Chen J G, Zhang S W. Liver cancer epidemic in China: past, present and future. Semin Cancer Biol. 2011; 21(1):59-69. Most HCC patients have rapid tumor progressing resulting in high mortality rate. In order to improve the overall survival, early diagnosis of the disease becomes essential. Currently, the most common way of detecting HCCs are blood tests that measure level of HCC tumor markers such as alpha fetoprotein (AFP). AFP is a plasma protein produced by yolk sac and liver during the development of fetus serving as a form of serum albumin. In normal condition, AFP level gradually decreases after birth and remain in low level in adults. Increased level of tumor markers indicates probability of liver cancers. However, the major problem of the AFP test is excessive false positives. It is because HCC is not the only cause for the AFP level elevation, but alcoholic hepatitis, chronic hepatitis or cirrhosis also associates with an increase of AFP.

Despite the fact that AFP test is commonly suggested for diagnosis of liver cancers, its result is not conclusive. Suspected patients will need to go through ultrasound imaging, CT scans or contrast MRI scans for further confirmation. Liver biopsy will be taken to distinguish whether the tumor is benign or malignant. However, conventional detection of HCCs comes with several limitations. About 20% of liver cancers do not produce elevated level of the commonly used HCC tumor markers. See Okuda K, Peters R L. Human alpha-1 fetoprotein. Hepatocellular Carcinoma. 1976:353-67. Viral cirrhosis produces false positive results on the blood tests. See Lok A S, Lai C L. Alpha-fetoprotein monitoring in Chinese patients with chronic hepatitis E virus infection: role in the early detection of hepatocellular carcinoma. Hepatology 1989; 9:110-115. Ultrasound is not able to detect small tumors. See Colombo M, de Franchis R, Del Ninno E, Sangiovanni A, De Fazio C, Tommasini M, Donato M F, Piva A, Di Carlo V, Dioguardi N. Hepatocellular carcinoma in Italian patients with cirrhosis. N Engl J Med. 1991; 325:675-80. CT scans require a high radiation dose and are insensitive to tumors less than 1 cm. See Sahani D V, Kalva S P. Imaging the Liver. The Oncologist. 2004; 9 (4): 385-397. MRI scans are expensive and the procedure is time consuming. Due to these limitations, there is a need to develop a novel HCC biomarkers screen with higher sensitivity and specificity for the purpose of early diagnosis of HCC and/or determining a prognosis of HCC to complement the conventional methods.

HCC tumor cells tend to produce a unique set of proteins when compared to the normal liver epithelial cells juxtaposed to the tumor. Evaluation of validated HCC tumor biomarkers has the great potential to facilitate the diagnosis of HCC. However, not all HCC biomarkers themselves can be found in serum or urine for convenient diagnosis. Alternatively, the auto-antibodies which are specifically against the HCC biomarkers provide an opportunity to evaluate the expression of the biomarkers. It has been demonstrated in many cancers that the presence of tumor biomarkers couples the production of auto-antibodies against these tumor antigens. See Masutomi K, Kaneko S, Yasukawa M, Arai K, Murakami S, Kobayashi K. Identification of serum anti-human telomerase reverse transcriptase (hTERT) auto-antibodies during progression to hepatocellular carcinoma. Oncogene. 2002 Aug. 29; 21(38):5946-50; Karanikas V, Khalil S, Kerenidi T, Gourgoulianis K I, Germenis A E. Anti-surviving antibody responses in lung cancer. Cancer Lett. 2009 Sep. 18; 282(2):159-66; Wang Y Q, Zhang H H, Liu C L, Xia Q, Wu H, Yu X H, Kong W. Correlation between auto-antibodies to survivin and MUC1 variable number tandem repeats in colorectal cancer. Asian Pac J Cancer Prey. 2012; 13(11):5557-62. Detection of auto-antibodies in patients' sera provides for a more efficient examination for the presence of biomarkers. Examination of auto-antibodies from peripheral blood provides for the early detection of liver cancers, and in a non-invasive manner. The present invention also supports high-throughput screening. This may alleviate the cost required for conventional liver cancer diagnosis.

### SUMMARY OF INVENTION

The present invention provides a detection and quantification method for measuring the auto-antibodies against a certain panel of specific tumor HCC biomarkers, which is useful for diagnosing and staging cancers. Comparing to normal liver epithelial cells, HCC tumor cells tend to produce a unique set of proteins. The evaluation of the unique protein set of HCC biomarkers complements conventional diagnostic methods and facilitates early detection of cancers.

By using a Two-Dimensional/Mass Spectrometry based method, a set of liver cancer HCC biomarkers from paired patients' biopsies (tumor biopsy versus juxtaposed normal tissue) was identified. The HCC biomarkers include Bmil, VCC1, SUMO-4, RhoA, TXN, ET-1, UBE2C, HDGF2 (which is also known as HDGFRP3), FGF21, LECT2, SOD1, STMN4, Midkine (known as MK), IL-17A, IL26 and DCP.

Specificity and accuracy of this set of liver cancer biomarkers was then validated and taken together for diagnosis of liver cancers. In the present invention, proteins of the above listed HCC biomarkers (and the HCC biomarkers in the HCC biomarker conjugates) were expressed from cDNA clones, purified and coupled to fluorescent microsphere beads with different emission wavelengths to make a protein-bead conjugate. Auto-antibodies present in patients' sera against the proteins immunologically bind to the protein-bead conjugate. The auto-antibodies subsequently interact with PE-conjugated secondary antibodies. The specific fluorescence signal of the microsphere beads serves as an identifier for the conjugated HCC biomarkers. By measuring the fluorescent intensity given by the PE-conjugated secondary antibodies at the complex, it allows the detection and quantification of the auto-antibodies. Since the auto-antibodies are produced in the patients' sera in proportion to the abundance of the HCC biomarkers at HCC tumor cells, the higher fluorescent intensity resulting from a higher concentration of auto-antibodies indicates the higher expression of the corresponding HCC biomarkers. The lowest detection limit of each HCC biomarker to the total serum auto-antibodies is about 0.15 ng/mL.

Comparing to sera from healthy subjects, the level of auto-antibodies against the target HCC biomarkers is at a higher concentration in a cancer patient. Moreover, comparing different sera from liver cancer patients at different stages, signature patterns for staging may be generated. Thus, the present invention allows the non-invasive evaluation of the targeted liver cancer biomarker. This enables the detection of HCC at early stages and the identification of signature HCC biomarker patterns for staging, as well as the detection of recurrences during a monitoring period of post-chemotherapeutic treatment.

### SEQUENCE LISTING

The instant application contains a Sequence Listing which has been submitted electronically in ASCII format and is hereby incorporated by reference in its entirety. Said ASCII copy, created on Jul. 21, 2020, is named 1H8220-000023_SL.txt and is 28,824 bytes in size.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present invention are described in more detail hereinafter with reference to the drawings, in which:

FIGS. 1A and 1B shows the difference in protein expression pattern between tumor biopsy (FIG. 1B) and juxtaposed normal tissue (FIG. 1A) by two-dimensional/mass spectrometry leading to the identification of 15 specific HCC biomarkers up-regulated in liver cancer; arrows indicate location of spots identified on a 2-D gel of the mass spectrometry.

FIG. 2 shows a set of 15 validated liver cancer biomarkers and their corresponding molecular weight targeted and measured in the present invention.

FIG. 3 shows the workflow of expressing the HCC biomarkers from cDNA clones.

FIG. 4 shows the workflow of purification of the HCC biomarkers expressed from E. coli.

FIG. 5 shows the workflow of measuring the auto-antibodies by Bioplex™ system.

FIG. 6 shows the conjugation of HCC biomarker protein to Bioplex™ bead.

FIG. 7 shows illustration of the complex of biomarker-Bioplex™ bead conjugate immunoreacting with HCC biomarker auto-antibody and PE-conjugated secondary antibody.

FIGS. 8A and 8B show the gel electrophoresis of the DNA insert released from plasmid cut by restriction enzymes HindIII (FIG. 8A) and BamH (FIG. 8B).

FIG. 9A through FIG. 9E show the Coomassie Blue stained SDS-PAGE verifying the IPTG induction of various HCC biomarkers. FIG. 9A shows Bmil; FIG. 9B shows SOD1; FIG. 9C shows IL-17A; FIG. 9D shows TXN; and FIG. 9E shows Midkine.

FIG. 10A through FIG. 10C show the elution profile of various HCC biomarkers in AKTA. FIG. 10A shows Bmil; FIG. 10B shows SOD-1; and FIG. 10C shows IL-17A.

FIG. 11A through FIG. 11C show the Coomassie Blue stained SDS-PAGE verifying the purification of His-tagged proteins; Fraction A is bacteria without IPTG induction; Fraction B is bacteria with IPTG induction; Fraction C is bacterial lysate. FIG. 11A shows Bmil; FIG. 11B shows SOD-1; and FIG. 11C shows IL-17A.

FIG. 12 shows the standard curve showing the fluorescence intensity against the concentration of anti-Bmil antibody.

FIG. 13 is a schematic diagram showing the design of the test: Patient serum containing auto-antibodies are mixed in a well containing 15 types of beads corresponding to 15 HCC biomarkers of the HCC biomarker set, followed by the addition of PE-conjugated secondary antibody.

FIG. 14A through FIG. 14H show various multiplex curves, which enable the calculation of the concentration of auto-antibodies present in the serum sample. The figures provide test results run against various HCC biomarkers. The curve is fit with five-parameter logistics (5 PL) and an equation corresponding to the curve is automatically generated by the Bioplex™ machine. By substituting Y value (PE signal) into the equation, the applicants were able to calculate the X value (auto-antibody concentration). FIG. 14A provides results of tests run against the MK (Midkine) biomarker. FIG. 14B provides results of tests run against the IL26 biomarker. FIG. 14C provides results of tests run against the IL-17A biomarker. FIG. 14D provides results of tests run against the RhoA biomarker. FIG. 14E provides results of tests run against the FGF21 biomarker. FIG. 14F provides results of tests run against the HDGFRP3 biomarker. FIG. 14G provides results of tests run against the SOD1 biomarker. FIG. 14H provides results of tests run against the TXN biomarker.

FIG. 15 shows an ET1 multiplex curve, which enables the calculation of the concentration of auto-antibodies present in the serum sample. This curve provides results of tests run against the ET-1 biomarker. The curve is fit with five-parameter logistics (5 PL) and an equation corresponding to the curve is automatically generated by the Bioplex™ machine. By substituting Y value (PE signal) into the equation, the applicants were able to calculate the X value (auto-antibody concentration).

FIGS. 16A-16I provide the results of studies of known HCC patients (Chinese), and classified as an “at risk group.” Applicants tested healthy and liver cancer samples to generate auto-antibody signals of each biomarker. They used internal serum positive and negative controls to determine assay variation, which was kept below 20% according to FDA guidelines. The auto-antibody signal was subtracted from the naked bead signal to minimize non-specific signal binding to the bead surface (i.e., to ensure that the signal reflects the auto-antibody binding to its corresponding biomarker). The results show that auto-antibodies were present in the HCC serum, where one dot represents one serum sample. FIG. 16A shows MK. FIG. 16B shows ET-1. FIG. 16C shows IL-26. FIG. 16D shows IL17A. FIG. 16E shows RhoA. FIG. 16F shows FGF21. FIG. 16G shows HDGFRP3. FIG. 16H shows SOD1. FIG. 16I shows TXN.

FIGS. 17A-17I provide the results of studies of known HCC patients (Caucasian and others), and classified as an “at risk group.” Applicants tested healthy and liver cancer samples to generate auto-antibody signals of each biomarker. They used internal serum positive and negative controls to determine assay variation, which was kept below 20% according to FDA guidelines. The auto-antibody signal was subtracted from the naked bead signal to minimize non-specific signal binding to the bead surface (i.e., to ensure that the signal reflects the auto-antibody binding to its corresponding biomarker). The results show that auto-antibodies were present in the HCC serum, where one dot represents one serum sample. FIG. 17A shows MK. FIG. 17B shows ET-1. FIG. 17C shows IL-26. FIG. 17D shows IL17A. FIG. 17E shows RhoA. FIG. 17F shows FGF21. FIG. 17G shows HDGFRP3. FIG. 17H shows SOD1. FIG. 17I shows TXN.

FIGS. 18A-18H provide the results of studies comparing healthy Chinese patients, against high alpha fetoprotein (AFP) levels and against low AFP levels. Applicants tested healthy and liver cancer samples to generate auto-antibody signals of each biomarker. They used internal serum positive and negative controls to determine assay variation, which was kept below 20% according to FDA guidelines. The auto-antibody signal was subtracted from the naked bead signal to minimize non-specific signal binding to the bead surface (i.e., to ensure that the signal reflects the auto-antibody binding to its corresponding biomarker). The results show that auto-antibodies were present in the HCC serum, where one dot represents one serum sample. FIG. 18A shows MK. FIG. 18B shows ET-1. FIG. 18C shows IL-26. FIG. 18D shows RhoA. FIG. 18E shows FGF21. FIG. 18F shows HDGFRP3. FIG. 18G shows SOD1. FIG. 18H shows TXN.

FIG. 19A shows the results of the DCP auto-antibody detection in sera from HCC patients compared to normal healthy (non HCC cancerous) sera in Asian patient samples. These results show that there is a 2-15 fold increase in signals between normal individuals and HCC patients. See example 5.

FIG. 19B shows the results of the DCP auto-antibody detection in sera from HCC patients compared to normal healthy (non HCC cancerous) sera in Caucasian patient samples. See example 5.

FIG. 20A through FIG. 20E show the Coomassie Blue stained SDS-PAGE verifying the IPTG induction of various HCC biomarkers. FIG. 20A shows HDGFRP3; FIG. 20B shows ET-1; FIG. 20C shows RhoA; FIG. 20D shows 11-26; and FIG. 20E shows FGF21.

FIG. 21A through FIG. 21G show the elution profile of various HCC biomarkers in AKTA. FIG. 21A shows MK; FIG. 21B shows ET-1; FIG. 21C shows IL-26; FIG. 21D shows RhoA; FIG. 21E shows FGF21; FIG. 21F shows HDGFRP3; and FIG. 21G shows TXN.

FIG. 22A shows the results of the measurement of auto-antibodies against IL26, HDGF2, IL17A and DCP in 40 healthy person samples and 40 HCC patient samples. The tests were done using the methods described herein—protein-bead conjugate and PE-conjugated secondary antibodies fed through the Bioplex™ machine. FIG. 22A provides the results for the healthy patients. Each “HEAxx” is a sample from an individual known to be a healthy person. The numbers in the table are the MFI (mean fluorescent intensity) measured from the PE-conjugated secondary antibodies showing the levels of auto-antibodies against the HCC biomarkers.

FIG. 22A shows the results of the measurement of auto-antibodies against IL26, HDGF2, IL17A and DCP in 40 healthy person samples and 40 HCC patient samples. The tests were done using the methods described herein—protein-bead conjugate and PE-conjugated secondary antibodies fed through the Bioplex™ machine. FIG. 22B provides the results for the HCC patient samples. Each “HCCxx” is a sample from an individual HCC patient. The numbers in the table are the MFI (mean fluorescent intensity) measured from the PE-conjugated secondary antibodies showing the levels of auto-antibodies against the HCC biomarkers.

FIG. 23 shows the Coomassie Blue stained SDS-PAGE verifying the purification of various His-tagged proteins. Fraction A is bacteria without IPTG induction; Fraction B is bacteria with IPTG induction.

### DETAILED DESCRIPTION OF INVENTION

In the following description, the HCC biomarker/biomarkers, the corresponding embodiments of the detection/validation/identification/quantification methods are set forth as preferred examples. It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

### Definitions

The term “biomarker” refers to the protein uniquely expressed or up-regulated in the tumor comparing to the normal epithelial cells.

The term “HCC biomarker set” refers to the specific combination of the HCC biomarkers identified from paired patients' biopsies (tumor biopsy versus juxtaposed normal tissue) and is the target of the measurement in the present invention.

The term “auto-antibodies” refers to the anti-bodies produced by the patient body coupling to the expression of the tumor biomarker and it is present in the circulation and can be collected in the peripheral serum.

Bmil (Polycomb Ring Finger) is a protein component of a Polycomb Group (PcG) multiprotein PRC1-like complex. It is responsible for maintaining the transcriptionally repressive state of many genes, including Hox genes, throughout development. The regulation is via monoubiquitination of histone H2A ‘Lys-119’, which modifies histone and remodels chromatin, rendering the expression.

VCC1 or CXCL17 (Chemokine (C-X-C Motif) Ligand 17) has an essential role in angiogenesis and possibly in the development of tumors. It is also suggested that it is a housekeeping chemokine regulating the recruitment of non-activated blood monocytes and immature dendritic cells into tissues. It may also play a role in the innate defense against infections. Malfunction of VCC1 is associated with duodenitis and cholera.

SUMO-4 (Small Ubiquitin-Like Modifier 4) belongs to the family of small ubiquitin-related modifiers and located in the cytoplasm. It covalently attaches to the target protein, IKBA, in order to control its subcellular localization, stability, or activity. This eventually leads to a negative regulation of NF-kappa-B-dependent transcription of the IL12B gene.

RhoA (Ras Homolog Family Member A) regulates the signaling pathway linking plasma membrane receptors to the assembly of focal adhesions and actin stress fibers. It also involves in microtubule-dependent signaling essential during cell cycle cytokinesis, and other signaling pathways involved in stabilization of microtubules and cell migrations and adhesion.

TXN (Thioredoxin) forms homodimer and is involved in redox reactions through the reversible oxidation of its active center dithiol to a disulfide and catalyzes dithiol-disulfide exchange reactions. It has been reported to be associated with breast mucinous carcinoma.

ET-1 (Endothelin 1) is a potent vasoconstrictor produced by vascular endothelial cells. It binds to endothelin receptors widely expressed in all tissues, including non-vascular structure like epithelial cells, glia, and neurons. Apart from the main role in maintenance of vascular tone, it is also suggested to have co-mitogenic activity and potentiate the effects of other growth factors.

UBE2C (Ubiquitin-Conjugating Enzyme E2C) belongs to the family of E2 ubiquitin-conjugating enzyme. This is one of the three enzymes involved in ubiquitination, which is an important cellular mechanism for targeting abnormal proteins for degradation. More specifically, UBE2C is required for the targeted degradation of mitotic cyclins and for cell cycle progression. Thus, it is believed that this protein may be also involved in cancer progression.

HDGF2 is called hepatoma-derived growth factor 2. This protein which is highly expressed in a variety of tumors has been reported to play a pivotal role in the development and progression of several tumors. Although the mechanism is yet to be identified, it is suggested that HDGF2 has mitogenic, angiogenic, neurotrophic and antiapoptotic activity. HDGF2 is also known as HDGFRP3.

FGF21 (Fibroblast Growth Factor 21) is a family member of the FGF family which is involved in vary biological processes including embryonic development, cell growth, morphogenesis, tissue repair, tumor growth and invasion. More specifically, FGF21 stimulates glucose update in differentiated adipocytes via the induction of glucose transporter SLC2A1/GLUT1 expression. It has been found that FGF21 is associated with fatty liver disease.

LECT2 (Leukocyte Cell Derived Chemotaxin 1) is a secretory protein acts as a chemotactic factor to neutrophils and stimulates the growth of chondrocytes and osteoblasts. This protein is associated with acute liver failure.

SOD1 (Superoxide Dismutase 1) is a Cu/Zn-containing antioxidant enzyme responsible for destroying free superoxide radicals into molecular oxygen and hydrogen peroxide in the cytosol, the nucleus, and the intermembrane space of the mitochondria. It is important for maintaining low levels of superoxide in the cytosol, thus protecting the cell from oxidative stress and subsequent cell death.

STMN4 (Stathmin-Like 4) is a small regulatory protein which is believed to have a role in relaying integrating diverse intracellular signaling pathways, which in turn, controls cell proliferation, differentiation and functions. It is also shown that this protein contributes to the control of microtubule dynamics by inhibiting the polymerization of microtubules and/or favoring their depolymerization.

Midkine or NEGF2 (Neurite Growth-Promoting Factor 2) is a secretory growth factor that binds heparin and responsive to retinoic acid. Midkine promotes cell growth, migration and angiogenesis, in particular during tumorigenesis. It has already been demonstrated to be associated with breast adenocarcinoma and soft tissue sarcoma.

IL-17A (Interleukin 17A) is a proinflammatory cytokine produced by the activated T cells. It regulates the activity of NF-kappaB and mitogen-activated protein kinases, stimulates the expression of IL6 and cyclooxygenase-2, and enhances the production of nitric oxide. Several chronic inflammation and sclerosis are usually associated with IL-17A elevation.

IL-26 (Interleukin 26) belongs to the IL-10 cytokine family and is produced by the activated T cells and targets epithelial cells for signal transduction. It binds strongly to glycosaminoglycans such as heparin, heparan sulphate, and dermatan sulfate on cellular surfaces which act similarly to co-receptors in order to enrich IL-26 on the surface of producer and target cells.

DCP (des-gamma-carboxy prothrombin) also known as the protein induced by vitamin K absence or antagonist II (PIVKA-II), is an abnormal form of the coagulation protein, prothrombin. DCP is a nonfunctional prothrombin resulting from a lack of carboxylation of 10 glutamic acid residues (amino acid residues 49, 50, 57, 59, 62-63, 68-69, 72 and 75) in the N-terminal portion of the molecule. In normal liver, prothrombin undergoes post-translational carboxylation before release into the peripheral blood. The carboxylation converts specific amino-terminal glutamic acid residues to gamma-carboxyglutamic acid. The vitamin K dependent carboxylase responsible for the carboxylation is absent in many hepatocellular carcinoma (HCC) cells, and an abnormal prothrombin with all or some of unconverted glutamic acid is secreted. Therefore, this noncarboxylated form (DCP) is used herein as an HCC biomarker. If one or more gamma-carboxylation sites of prothrombin is lacking at amino acid residues 49, 50, 57, 59, 62-63, 68-69, 72 or 75, the DCP auto-Ab can be detected by the present invention.

DCP is one of the tumor antigens present in HCC patients and so HCC patients will have auto-antibodies against DCP depending on the DCP variants they have in their bodies. The number of decarboxylation sites is an important consideration:

a. Current research suggests that control samples without HCC are expected to have no decarboxylation of the protein.

b. Benign tumor is expected to have more than 2 decarboxylation sites.

c. HCC patients are expected to have more than 5 decarboxylation sites. Therefore, in certain embodiments, the number of decarboxylation sites is an important consideration for screening potential HCC patients. A more robust screening that will likely not include false positives will focus on DCPs that have at least 5 decarboxylation sites. In some embodiments, the DCP has all 10 sites decarboxylated, or has greater than 5, greater than 6, greater than 7, greater than 8, or greater than 9 sites decarboxylated. In certain embodiments the DCP will have 1-10 decarboxylations, 2-10, 3-10, 4-10, 5-10, 6-10, 7-10, 8-10, 9-10, 5-10, 6-10, and 7-10 decarboxylations. The location of the decarboxylation site is not that important but generally occurs at residues 49, 50, 57, 59, 62-63, 68-69, 72 or 75.

The present inventors used a DCP recombinant protein produced from bacteria, which are unable to perform carboxylation. Thus, the resulting DCP protein has all 10 sites decarboxylated. Thus it is expected that this DCP protein has the strongest binding affinity to auto-antibodies against 9-10 decarboxylation sites; moderate binding to auto-antibodies against 6-8 decarboxylation sites; and very weak or no binding against auto-antibodies against 0-5 decarboxylation sites.

Regarding which sites are decarboxylated, publications suggest that common HCC decarboxylation sites are at amino acid position 59, 68-69 and 72.

Therefore, it is expected that there will be different auto-antibodies against all DCP variants; and the present invention screening test is expected to work for the most common DCP variants in HCC samples. In one embodiment, one DCP biomarker-Bioplex™ bead conjugate is made and is expected to work well with auto-antibodies against 6-10 decarboxylation sites, which are the most common DCP variants in HCC patients. The present inventors believe they are the first group to find an auto-antibody against DCP and realize that it can be a biomarker for HCC screening. Together with the other auto-antibodies against the HCC biomarkers discussed herein, the HCC screening test is more specific and sensitive than other HCC screenings available.

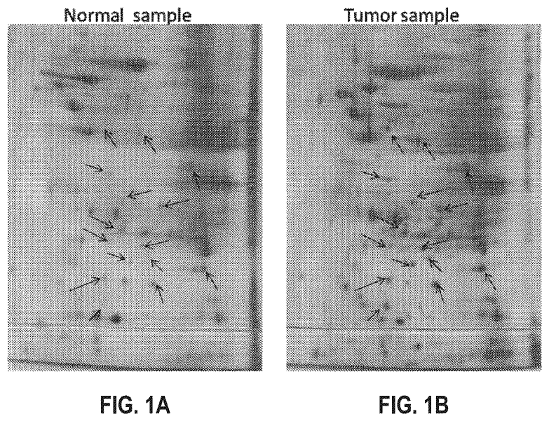

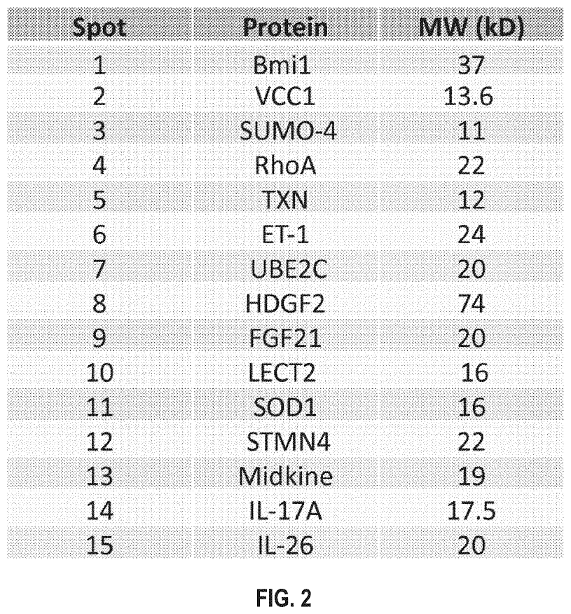

In the present invention, a set of 15 liver tumor biomarkers for detection and quantification of liver cancer was first identified by two-dimensional/mass spectrometry resolving the difference in the pattern of proteins expression between the paired patients' biopsies (tumor biopsy versus juxtaposed normal tissue) (FIG. 1). The HCC biomarkers were validated by immunohistochemical staining on paraffin-sectioned HCC blocks, and Western Blotting in HCC patients' sera. This resulted in a finalized list of 15 HCC biomarkers for the liver cancer diagnosis (FIG. 2). In addition, a novel HCC biomarker DCP is included in this set to account for 16 HCC biomarkers.

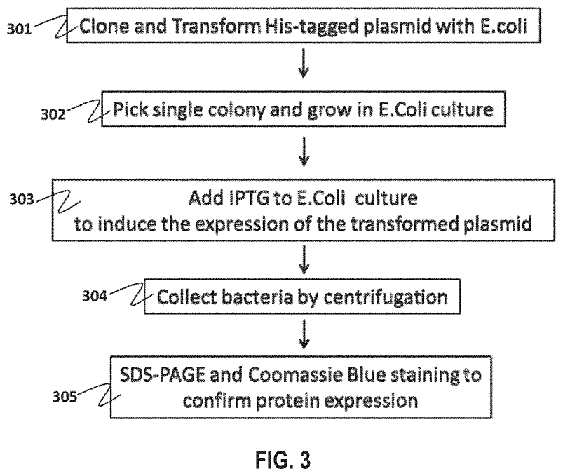

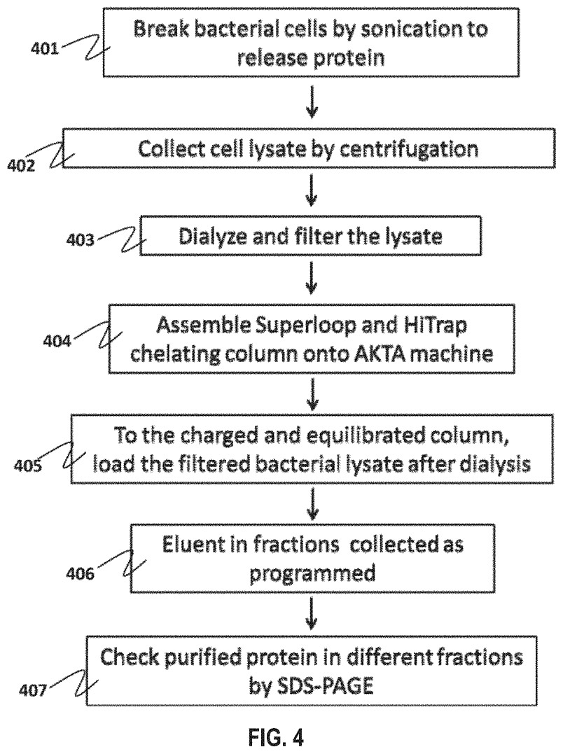

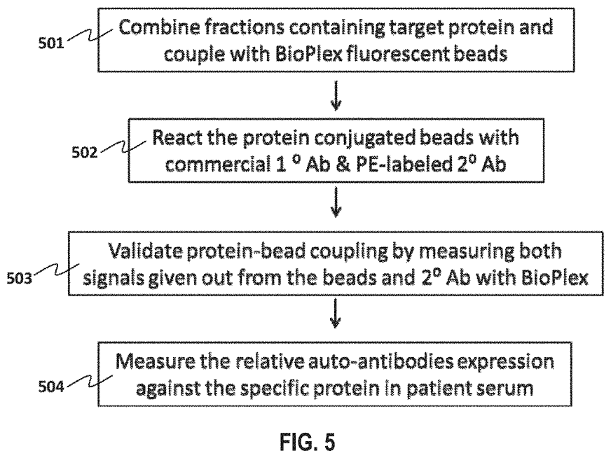

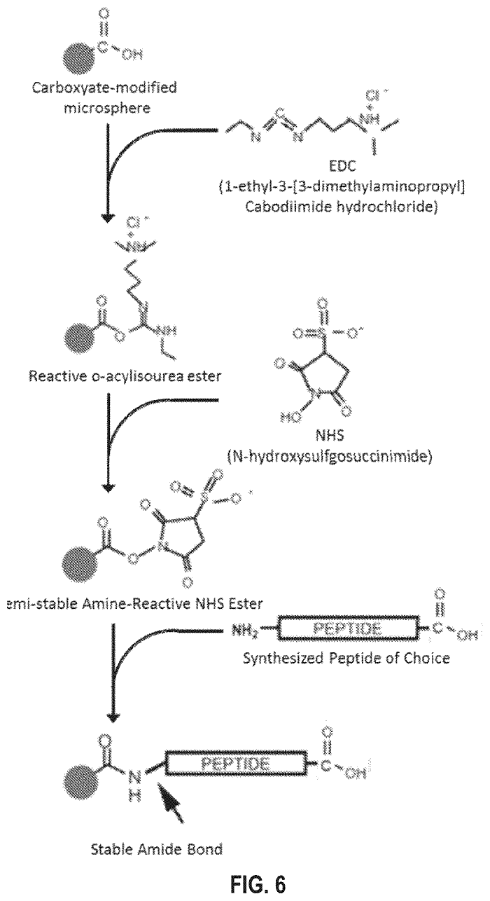

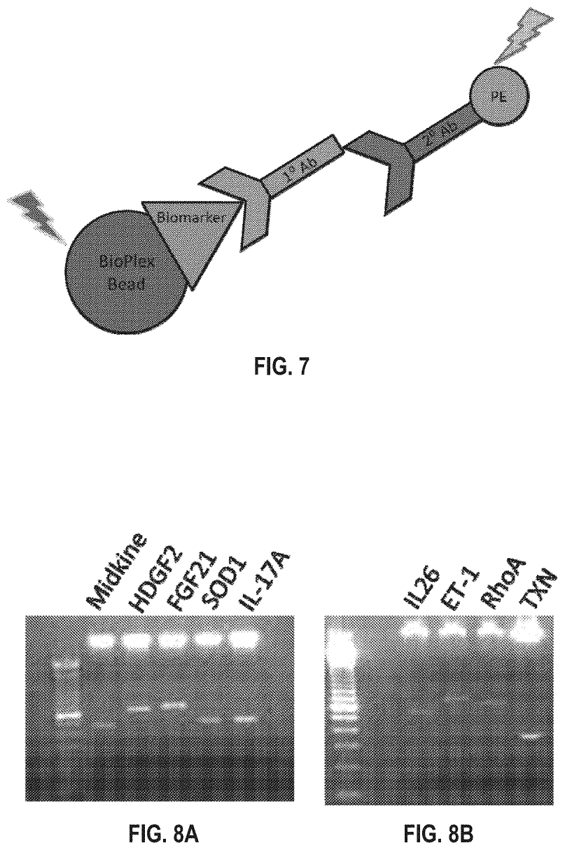

Based on the amino acid sequences of the targeted HCC biomarkers, commercially synthesized cDNA clones were employed for the expression of the HCC biomarker set (FIG. 3). Proteins expressed from the cDNA clones were then subjected to a series of steps of purifications (FIG. 4). The purified HCC biomarkers were subsequently conjugated via stable amide bonds with Bioplex™ beads (FIGS. 5, 6), a type of fluorescent microsphere beads and available in a panel which give unique fluorescent signals individually for identification at a multiplex set up. The HCC biomarkers on the beads are recognized by the specific HCC biomarker auto-antibodies, which are subsequently bound by an anti-human secondary antibody conjugated with PE (FIG. 7). Thus, the Bioplex™ machine simultaneously measures two signals from the complex. The fluorescence given by the Bioplex™ beads serves as an identifier, while the signal from the PE indicates the presence of the HCC biomarker in the complex. This also helps differentiating the protein-bead conjugates bound by the anti-body cascade from those with no immuno-reactivity with antibodies.

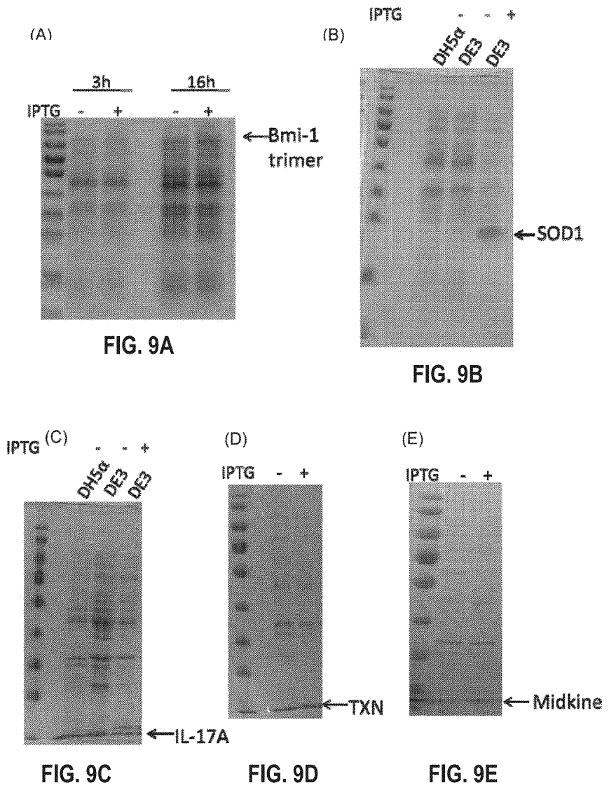

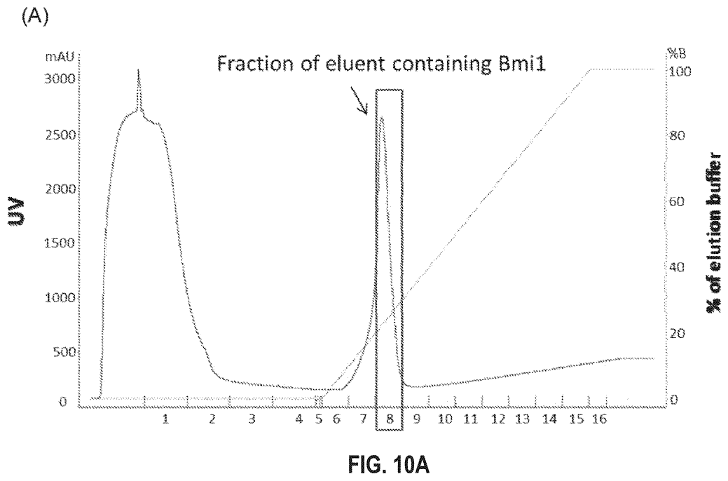

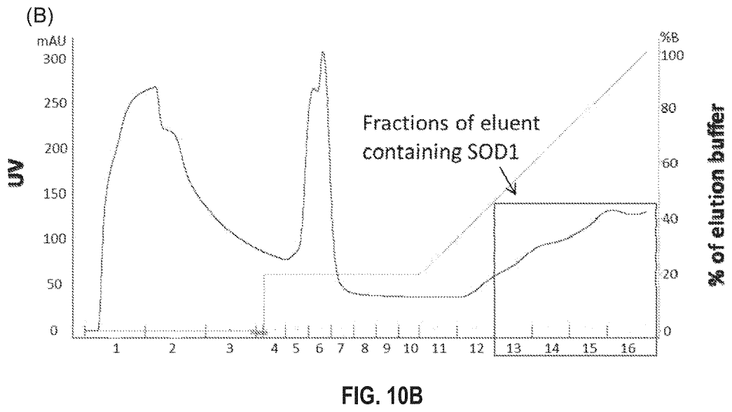

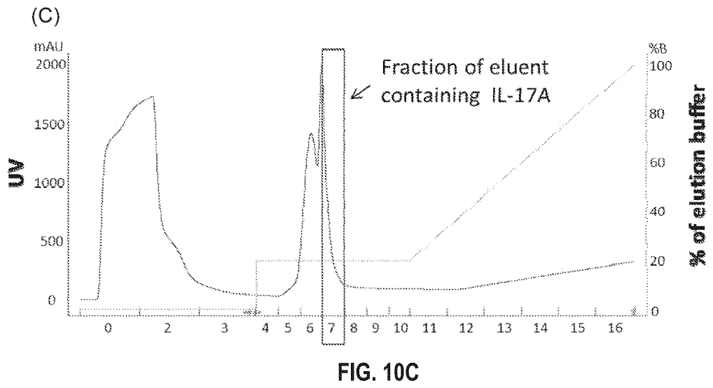

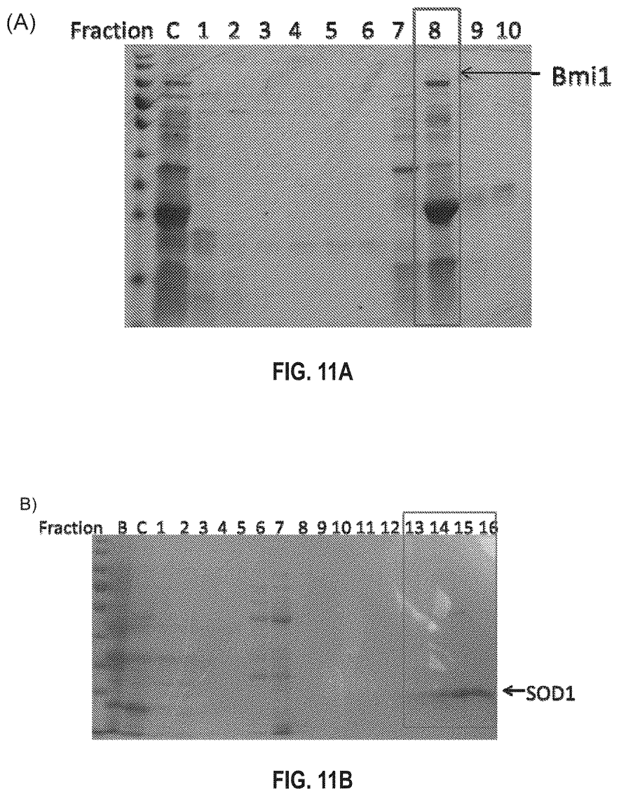

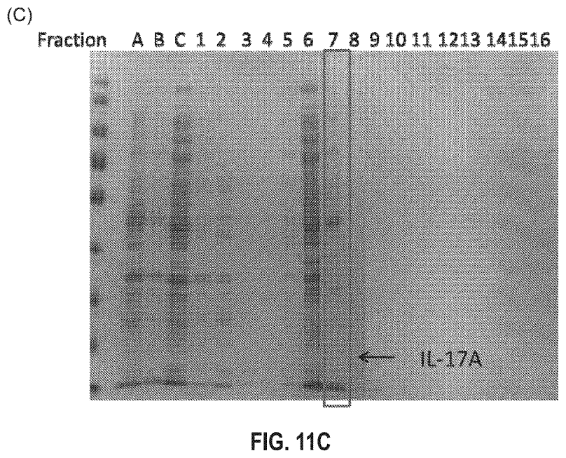

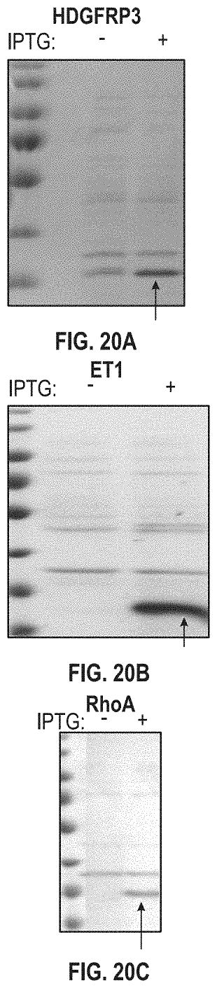

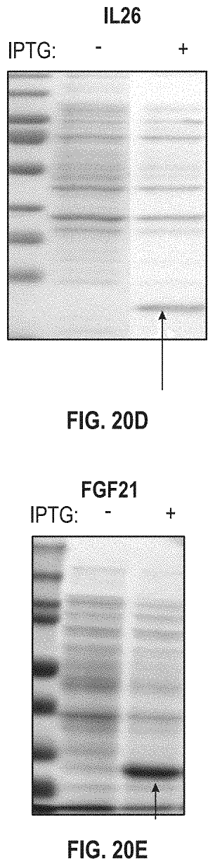

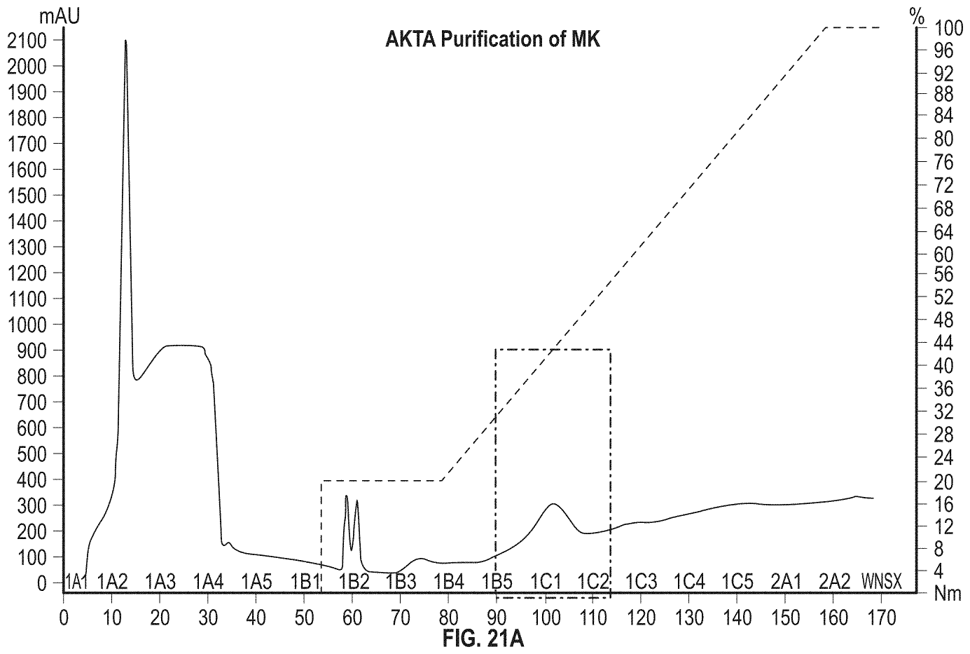

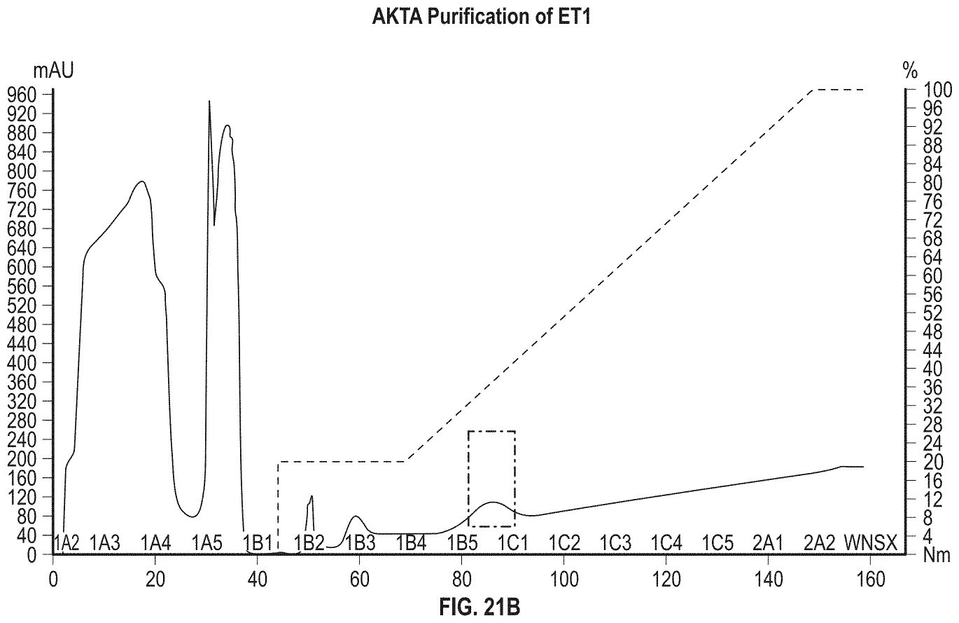

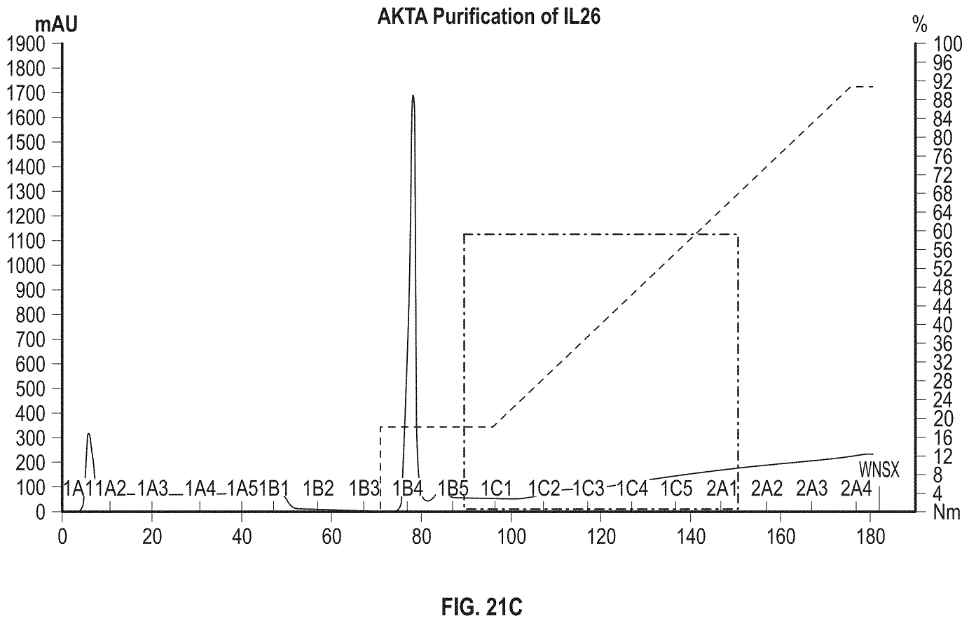

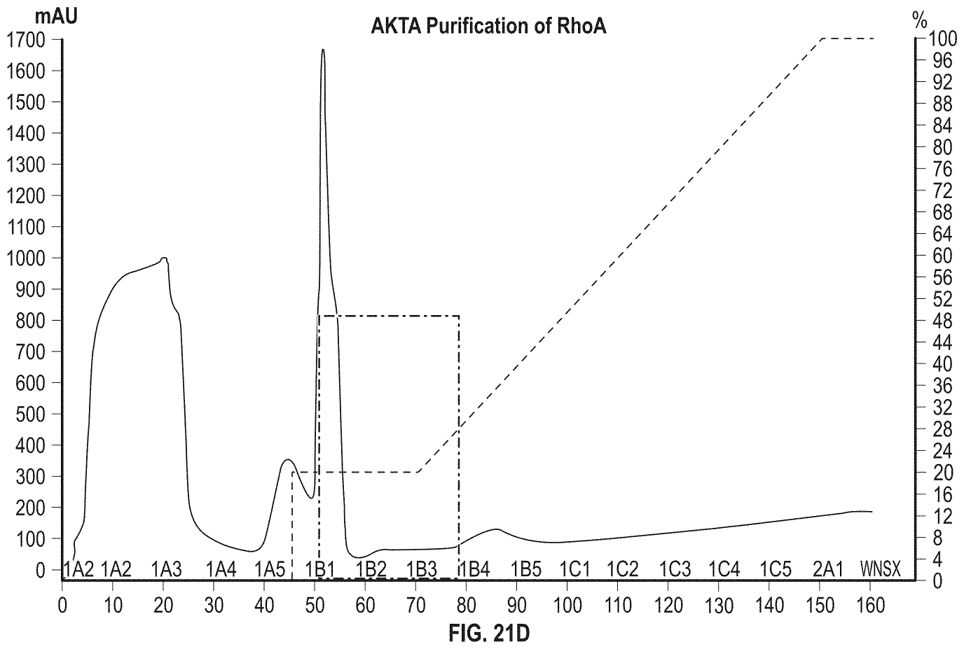

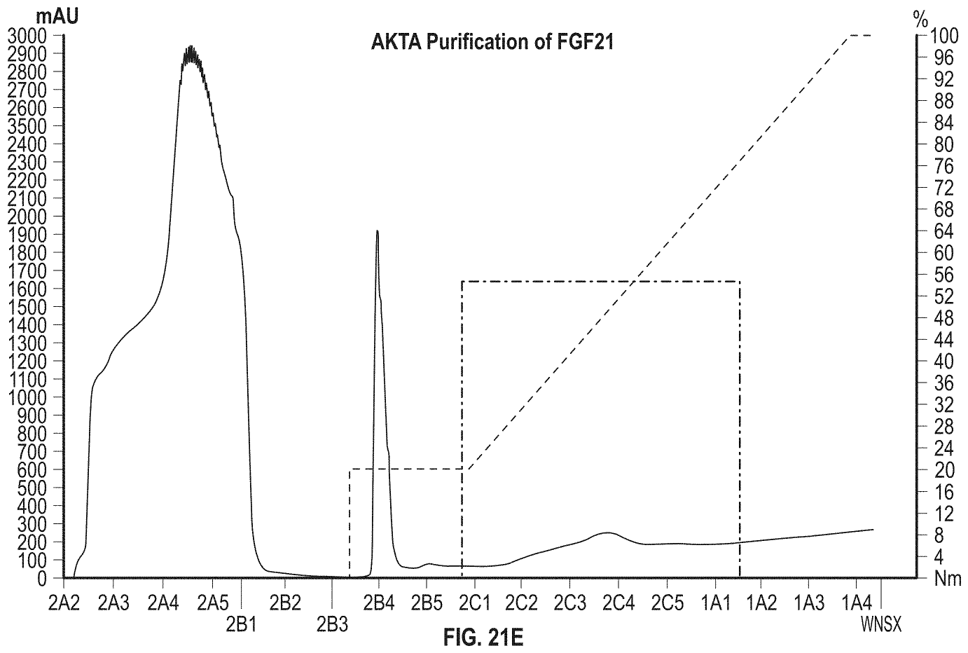

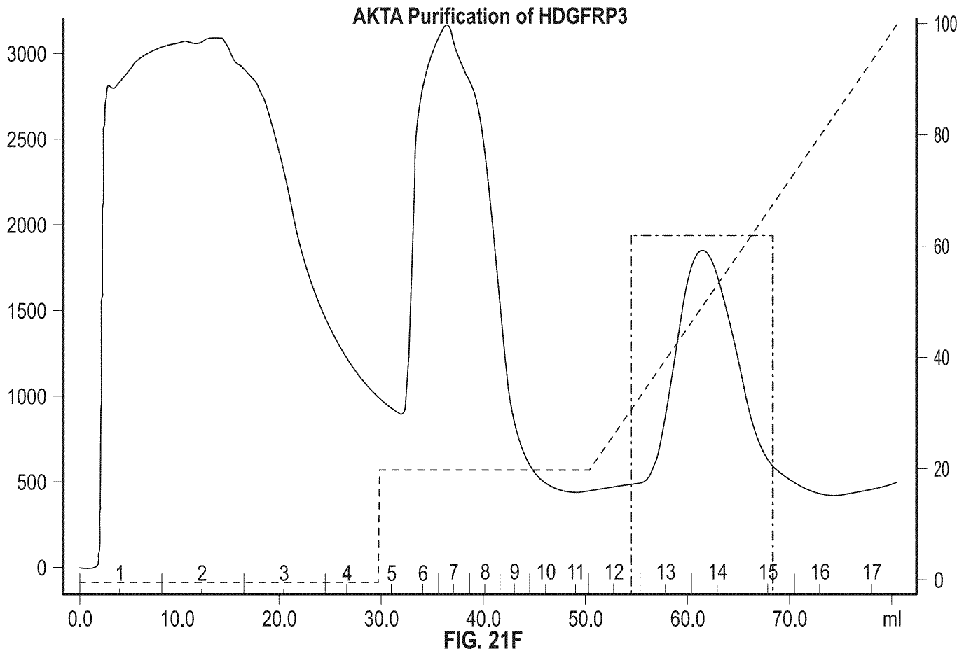

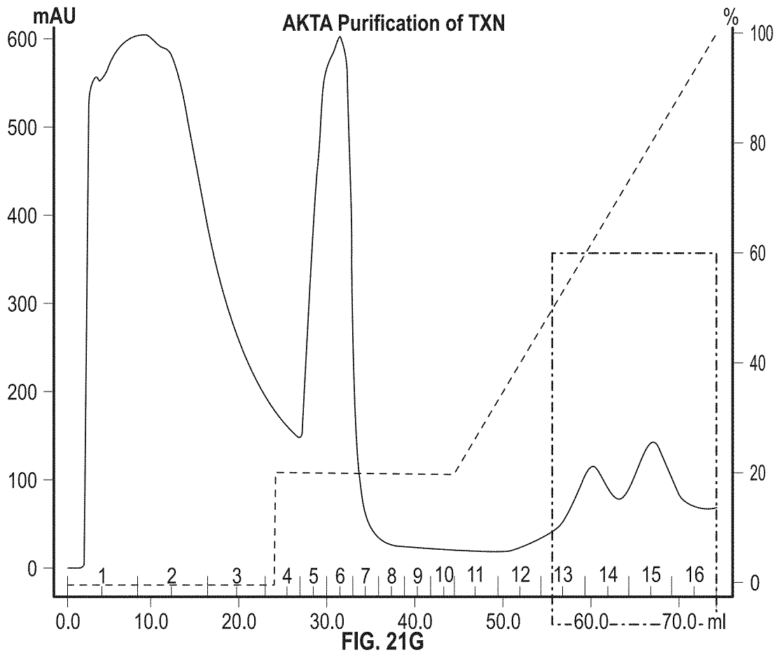

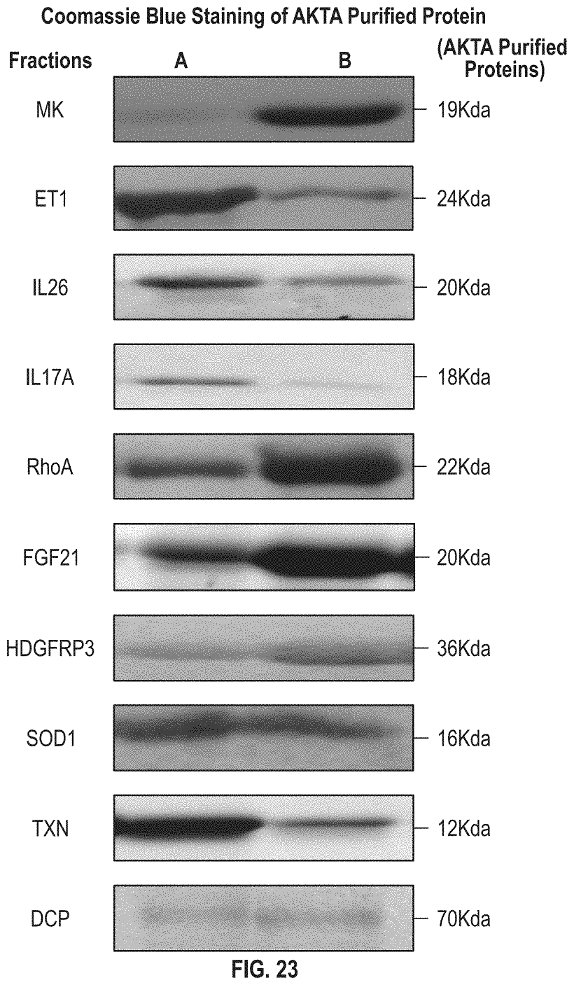

To prove the significance of the HCC biomarkers in the present invention, the cDNA clones were confirmed by restriction enzyme cut (FIGS. 8A and 8B). The transformed bacteria was induced by IPTG to express the HCC biomarker proteins. The protein expression was verified by SDS-PAGE and Coomassie Blue staining reveals the protein bands (FIG. 9A through FIG. 9E)(FIG. 20A-FIG. 20E). The His-tagged Bmil, SOD1 and IL-17A proteins were purified by AKTA (FIG. 10A-FIG. 10C) and then verified by SDS-PAGE and Coomassie Blue staining (FIG. 11A-FIG. 11C). The His-tagged MK (FIG. 21A), ET1 (FIG. 21B), IL-26 (FIG. 21C), RhoA (FIG. 21D), FGF21 (FIG. 21E), HDGFRP3 (FIG. 21F), and TXN (FIG. 21G) were purified by AKTA (FIG. 21A-FIG. 21G) and then verified by SDS-PAGE and Coomassie Blue staining) (FIG. 23) including the DCP biomarker.

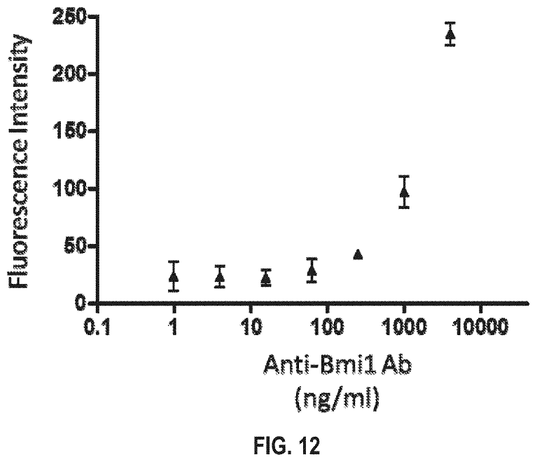

Sensitivity of the test was measured by spiking in a serial dilution of the antibodies. The lowest concentration of the antibody added that can give signal suggest the sensitivity of that particular biomarker. Meanwhile a standard curve was constructed showing the fluorescence intensity of the PE against the serial dilutions of the antibodies (FIG. 12). The standard curve was used for estimating the concentration of the HCC biomarker specific auto-antibodies in the patient sera by comparing the PE intensity.

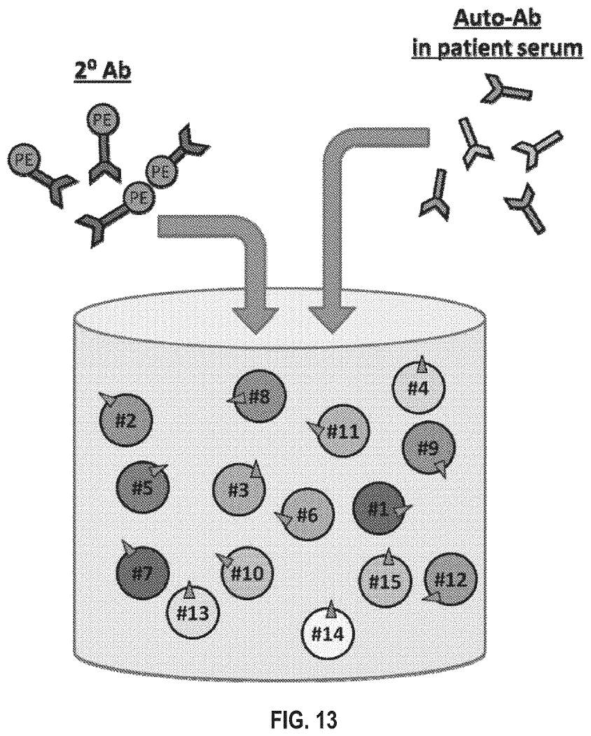

In the present invention, a multiplex of 16 different Bioplex™ beads individually giving unique fluorescence are conjugated with the HCC biomarker set and preloaded in the wells of a plate (FIG. 13—showing only the first original 15 HCC biomarkers). To a well, patient serum containing auto-antibodies is loaded and allowed to interact with the HCC biomarker conjugates. The PE-conjugated secondary antibodies are then added and bind to the auto-antibodies. In the machine, the excess secondary antibodies are washed away, the complex comprising the protein-bead conjugate and cascade of antibodies are measured individually. The unique fluorescence signal of the Bioplex™ bead identifies the HCC biomarkers, while the PE signal from the same complex indicates the presence of the HCC biomarker auto-antibodies (FIG. 7). Taken together, the measurement provides information about the presence of auto-antibodies and the relative concentration in the patients' sera.

In a standard randomized trial design, the mean of the relative level of auto-antibodies between the healthy group and patients diagnosed with liver cancer was compared. Student T test was used to analyze the variation significance. The significant difference indicates that the HCC biomarker is specific for liver cancer. After the verification trials, ranges of the concentration of HCC biomarker specific auto-antibodies will be obtained for the liver cancer positive and negative patients and serve as reference point for the future diagnosis. Meanwhile, expression pattern of the auto-antibodies was also compared between liver cancer patients of different stages. The signature patterns of the HCC biomarker expressions indicates the HCC staging.

Taken together, the measurement of the relative auto-antibodies level and the expression pattern of the HCC biomarkers, the present invention represents a different avenue to complement conventional liver cancer diagnosis. The present invention further enables non-invasive detection of auto-antibodies against the validated targets in patients' sera of the present invention, identifying the extent and the characteristics of the disease. Auto-antibodies naturally occur as a heterogeneous mixture even against one protein. There are two types of auto-antibodies. The first type is to recognize protein by short amino acid sequence only. The second type is to recognize protein by protein structure and also short amino acid sequence. Both types of auto-antibodies occur naturally in human to target even one protein.

Apart from early detection for stage I liver cancers, the present invention also enables the generation of signature patterns for staging, and the detection of recurrences during a monitoring period of post-mastectomy or post-chemotherapeutic treatment.

Thus in certain embodiments, there is provided a method for measuring the presence of hepatocellular carcinoma (HCC) biomarkers (indirectly by measuring the presence of auto-antibodies to the HCC biomarkers) in a subject suspected of having HCC. Methods comprise obtaining a serum sample from the subject suspected of having HCC and measuring the serum for the presence of auto-antibodies against a set of HCC biomarkers. The set of HCC biomarkers includes the following HCC biomarkers: Bmi-1, VCC1, SUMO-4, RhoA, TXN, ET-1, UBE2C, HDGF2, FGF21, LECT2, SOD1, STMN4, Midkine, IL-17A, IL26 and DCP.

In certain embodiments, the serum is measured for the presence of auto-antibodies to all 16 of the aforementioned HCC biomarkers. In certain embodiments, the serum is measured for the presence of auto-antibodies to at least one of these HCC biomarkers. In certain embodiments, the serum is measured for the presence of auto-antibodies to at least the DCP biomarker. In certain embodiments, the serum is measured for the presence of auto-antibodies to the DCP biomarker and at least one other biomarker selected from the group consisting of: Bmi-1, VCC1, SUMO-4, RhoA, TXN, ET-1, UBE2C, HDGF2, FGF21, LECT2, SOD1, STMN4, Midkine, IL-17A, and IL26.

In certain embodiments, the serum is measured for the presence of auto-antibodies to the following seven HCC biomarkers: DCP, IL26, RhoA, HDGF2, SOD1, TXN and IL-17A.

In certain embodiments, the serum is measured for the presence of auto-antibodies to only the following seven HCC biomarkers: DCP, IL26, RhoA, HDGF2, SOD1, TXN and IL-17A.

In certain embodiments, the serum is measured for the presence of auto-antibodies to the following seven HCC biomarkers: DCP, IL26, RhoA, HDGF2, SOD1, TXN and IL-17A and optionally to at least one other HCC biomarker selected from the group consisting of Bmi-1, VCC1, SUMO-4, ET-1, UBE2C, FGF21, and SOD1.

In certain embodiments, the serum is measured for the presence of auto-antibodies to the DCP biomarker and at least one other HCC biomarker selected from the group consisting of: Midkine, IL26, IL17A, RhoA, HDGF2, SOD1, and TXN.

In certain embodiments, the serum is measured for the presence of auto-antibodies to the DCP biomarker and any one of the following HCC biomarkers: Bmi-1, VCC1, SUMO-4, RhoA, TXN, ET-1, UBE2C, HDGF2, FGF21, LECT2, SOD1, STMN4, Midkine, IL-17A, and IL26.

In certain embodiments, the serum is measured for the presence of auto-antibodies to the DCP biomarker and any number of or combination of the following HCC biomarkers: Bmi-1, VCC1, SUMO-4, RhoA, TXN, ET-1, UBE2C, HDGF2, FGF21, LECT2, SOD1, STMN4, Midkine, IL-17A, and IL26.

In other embodiments, the serum is measured for the presence of auto-antibodies to the at least following 8 HCC biomarkers: Midkine, IL26, IL17A, RhoA, HDGF2, SOD1, TXN and DCP.

In other embodiments, the serum is measured for the presence of auto-antibodies to the following 8 HCC biomarkers: Midkine, IL26, IL17A, RhoA, HDGF2, SOD1, TXN and DCP.

In other embodiments, the serum is measured the presence of auto-antibodies to only the following 8 HCC biomarkers: Midkine, IL26, IL17A, RhoA, HDGF2, SOD1, TXN and DCP.

In other embodiments, the serum is measured for the presence of auto-antibodies to the only the following 16 HCC biomarkers: Bmi-1, VCC1, SUMO-4, RhoA, TXN, ET-1, UBE2C, HDGF2, FGF21, LECT2, SOD1, STMN4, Midkine, IL-17A, IL26 and DCP.

In other embodiments, the serum is measured for the presence of auto-antibodies against the HCC biomarkers comprises measuring for the presence of auto-antibodies to DCP and measuring for the presence of auto-antibodies to Bmi-1, VCC1, SUMO-4, RhoA, TXN, ET-1, UBE2C, HDGF2, FGF21, LECT2, SOD1, STMN4, Midkine, IL-17A, and IL26.

In other embodiments, the serum is measured for the presence of auto-antibodies against the HCC biomarkers comprises measuring for the presence of auto-antibodies to DCP and measuring for the presence of auto-antibodies to RhoA, TXN, HDGF2, SOD1, IL-17A, and IL26.

In other embodiments, the serum is measured for the presence of auto-antibodies against the HCC biomarkers comprises measuring for the presence of auto-antibodies to DCP and measuring for the presence of auto-antibodies to at least one of RhoA, TXN, HDGF2, SOD1, IL-17A, and IL26.

In other embodiments, the serum is measured for the presence of auto-antibodies against the HCC biomarkers comprises measuring for the presence of auto-antibodies to DCP and auto-antibodies to HDGF2, IL-17A, and IL26.

In other embodiments, the serum is measured for the presence of auto-antibodies against the HCC biomarkers comprises measuring for the presence of auto-antibodies to DCP and auto-antibodies to at least one of HDGF2, IL-17A, and IL26.

In other embodiments, the serum is measured for the presence of auto-antibodies against the HCC biomarkers comprises measuring for the presence of auto-antibodies to DCP and HDGF2.

In other embodiments, the serum is measured for the presence of auto-antibodies against the HCC biomarkers comprises measuring for the presence of auto-antibodies to DCP and HDGF2 and auto-antibodies to at least one of RhoA, TXN, SOD1, IL-17A, and IL26.

In other embodiments, the serum is measured for the presence of auto-antibodies against the HCC biomarkers comprises measuring for the presence of auto-antibodies to DCP and HDGF2 and auto-antibodies to at least one of IL-17A, and IL26.

Detecting the presence of the HCC biomarkers (by detecting the presence of HCC biomarker auto-antibodies) in the subject suspected of having HCC involves the following steps:

a. obtaining a serum sample from the subject suspected of having HCC and measuring the serum for the presence of auto-antibodies against a set of HCC biomarkers, wherein the set of HCC biomarkers includes DCP and at least one other HCC biomarker selected from the group consisting of Bmi-1, VCC1, SUMO-4, RhoA, TXN, ET-1, UBE2C, HDGF2, FGF21, LECT2, SOD1, STMN4, Midkine, IL-17A, and IL26;

b. detecting the presence of the HCC biomarkers in the subject suspected of having HCC, the method comprising the steps of:
i. mixing the serum sample with a set of HCC biomarker conjugates to allow the auto-antibodies to the HCC biomarkers, if present in the serum sample, to bind to a set of HCC biomarker conjugates and washing away any unbound auto-antibodies;

wherein the set of HCC biomarker conjugates comprises each of the HCC biomarkers in the set of HCC biomarkers conjugated via an amide bond to a unique fluorescent microsphere bead,

wherein each unique fluorescent microsphere bead associated with a specific particular HCC biomarker in the set of HCC biomarkers has a different emission wavelength for each HCC biomarker,

wherein the HCC biomarker conjugates are capable of being bound by a specific auto-antibody against an HCC biomarker present in the subject's serum sample,
ii. adding to the mixture formed in step i. an anti-human secondary antibody conjugated with phycoerythrin (PE), which is capable of binding the auto-antibodies to the HCC biomarkers; and allowing the anti-human secondary antibody conjugated with PE to bind to specific auto-antibodies that are bound to HCC biomarker conjugates to form a fluorescent bead-biomarker-auto antibody-PE conjugated antibody cascade, and washing away an unbound anti-human secondary antibody; and
iii. measuring the mixture formed in step ii. for the presence of the fluorescent bead-biomarker-auto antibody-PE conjugated antibody cascade to determine whether the subject's serum contained auto-antibodies to the HCC biomarkers.

The presence of the HCC biomarker auto-antibodies is useful to determine whether the subject has HCC and/or determine the stage of the cancer.

The fluorescent intensity given by the PE-conjugated secondary antibodies in the fluorescent bead-biomarker-auto antibody-PE conjugated antibody cascade can be measured to allow the detection and quantification of the HCC biomarker auto antibodies.

In certain embodiments the set of HCC biomarkers can be any one of the sets described above or can be an individual HCC biomarker as described above.

Methods of the invention also provide for measuring the presence of hepatocellular carcinoma (HCC) biomarkers (indirectly by measuring the presence of HCC biomarker auto-antibodies) in a plurality of subjects having HCC at different stages.

When looking at the staging of the cancer, methods involve comparing the level of HCC biomarker auto-antibodies measured in sera from the plurality of HCC patients who have different HCC cancer stages to generate a signature pattern of either the HCC biomarker expression levels or the HCC biomarker auto-antibody levels in the patient's sera for each different stage of HCC cancer to generate a HCC biomarker or HCC biomarker auto-antibody profile for each stage of HCC cancer.

Also provided herein are kits for detecting HCC biomarker auto-antibodies to a plurality of hepatocellular carcinoma (HCC) biomarkers in a patient's serum. The kits may comprise any one of the HCC biomarker conjugates, which conjugates comprise an HCC biomarker protein discussed herein (Bmi-1,. VCC1, SUMO-4, RhoA, TXN, ET-1, UBE2C, HDGF2, FGF21, LECT2, SOD1, STMN4, Midkine, IL-17A, IL26 and DCP) including the proteins set forth in SEQ ID NOs: 1-16. Each HCC biomarker protein is coupled to a different fluorescent microsphere bead having a different emission wavelength. The kits may contain a PE-conjugated secondary antibody capable of binding to all of the HCC biomarker auto-antibodies wherein the HCC biomarker auto-antibodies are to the HCC biomarker proteins Bmi-1, VCC1, SUMO-4, RhoA, TXN, ET-1. UBE2C, HDGF2, FGF21, LECT2, SOD1, STMN4, Midkine, IL-17A, IL26 and DCP including the proteins set forth in SEQ ID NOs: 1-16.

The kit is preferably capable of detecting an auto-antibody to any one of the hepatocellular carcinoma (HCC) biomarkers present in the patient's serum when the auto-antibody is present at an amount as low as about 0.15 ng/mL.

Also provided herein is a kit for detecting auto-antibodies to a plurality of hepatocellular carcinoma (HCC) biomarkers in a patient's serum, where the kit comprises:

a. a set of hepatocellular carcinoma (HCC) biomarker conjugates comprising HCC biomarker proteins HDGF2 and DCP wherein each HCC biomarker protein is coupled to a different fluorescent microsphere bead having a different emission wavelength; and

b. a PE-conjugated secondary antibody capable of binding to the HCC biomarker auto-antibodies, wherein the HCC biomarkers auto-antibodies are to the HCC biomarker proteins HDGF2 and DCP.

Also provided is a kit as described above that also includes at least one additional HCC biomarker protein conjugate comprising HCC biomarker proteins selected from the group consisting of Bmi-1, VCC1, SUMO-4, RhoA, TXN, ET-1, UBE2C, FGF21, LECT2, SOD1, STMN4, Midkine, IL-17A, and IL26; and

b. a PE-conjugated secondary antibody capable of binding to the HCC biomarker auto-antibodies, wherein the HCC biomarker auto-antibodies are to the HCC biomarker proteins HDGF2 and DCP.

Also provided herein is a kit wherein the set of set of hepatocellular carcinoma (HCC) biomarker conjugates comprises:

c) at least one additional HCC biomarker protein conjugate comprising HCC biomarker proteins selected from the group consisting of Bmi-1, VCC1, SUMO-4, RhoA, TXN, ET-1, UBE2C, FGF21, LECT2, SOD1, STMN4, Midkine, IL-17A, and IL26; and

In another embodiment the kit further comprises: a PE-conjugated secondary antibody capable of binding to the HCC biomarker auto-antibodies, wherein the HCC biomarker auto-antibodies are to the HCC biomarker proteins Bmi-1, VCC1, SUMO-4, RhoA, TXN, ET-1, UBE2C, FGF21, LECT2, SOD1, STMN4, Midkine, IL-17A, and IL26.

In another embodiment the kits described above may comprise a PE-conjugated secondary antibody capable of binding to all of the HCC biomarker auto-antibodies, wherein the HCC biomarker auto-antibodies are to the HCC biomarker proteins DCP, HDGF2, Bmi-1, VCC1, SUMO-4, RhoA, TXN, ET-1, UBE2C, FGF21, LECT2, SOD1, STMN4, Midkine, IL-17A, and IL26.

In any of the kits the DCP protein in the HCC biomarker conjugates was produced from bacteria that are unable to perform carboxylation of the DCP protein thereby expressing a DCP protein having all 10 sites decarboxylated.

### EXAMPLES

The following examples are provided by way of describing specific embodiments of this invention without intending to limit the scope of this invention in any way.

### Example 1a: Protein Extraction from Patients' Biopsies

500 mg of the paired patients' biopsies (tumor biopsy versus juxtaposed normal tissue) are collected and washed with PBS. The tissues were frozen by submerging into liquid nitrogen and immediately homogenized with pestle and mortar. To the homogenized samples, lysis solution (8M Urea, 4% CHAPS, 2% IPG Buffer, 0.2 mg/ml PMSF) was added, then vortex for at least 5 min until the tissues are completely dispersed. The lysates were then clarified by centrifugation at 14,000 rpm for 10 minutes at 4° C. The supernatants were further cleaned up by 2D Clean Up kit (Amersham) to remove the salt and impurities. The pellets were resuspended with minimum volume of Rehydration Solution (No DTT & IPG Buffer added). The protein concentrations were then measured by Bio-Rad™ protein assay and aliquots of 200 g/per tube are stored at −70° C.

### Example 1b: Resolving Proteins by Two-Dimensional Electrophoresis

To 1 ml rehydration stock solution, 2.8 mg DTT, 5 μl pharmalyte or IPG Buffer, and 2 μl bromophenol blue was added. 50-100 μg of protein sample is added to the 13 cm Immobiline DryStrip™ (IPG strip) containing 250 μl of rehydration solution. After removing the protective cover, the IPG strip was positioned in the strip holder with the gel side facing down, and overlaid with Cover Fluid to prevent dehydration during electrophoresis. The strip was then placed on to Ettan™ IPGphor™ (Amersham) for isoelectric focusing (first dimensional electrophoresis).

After the first-dimensional electrophoresis, the IPG strip was equilibrated with equilibrate solution (6 M Urea, 2% SDS, 50 mM Tris HCl pH 6.8, 30% Glycerol, 0.002% Bromophenol blue, 100 mg DTT per 10 ml buffer and 250 mg IAA per 10 ml buffer), and then washed with 1×SDS running Buffer for 4-5 times. The IPG strip was placed on top of the second-dimension gel and overlaid with sealing solution (0.5% Low Melting agarose, 0.002% Bromophenol Blue in 1×SDS running Buffer). The second-dimensional electrophoresis was then carried out at 30 mA for first 15 min followed by 60 mA for 3-4 h.

Upon the completion of the second dimensional electrophoresis, the gel is removed from the cassette, fixed and stained with silver nitrate. 15 spots representing 15 up-regulated proteins are identified (FIG. 1). To identify the proteins (FIG. 2), the silver stained gel slices were destained and trypsinized to release the protein from the gel for MALDI-TOF analysis.

### Example 2a: Expression of HCC Biomarker Set

His tagged plasmids containing cDNA inserts encoding the HCC biomarker set was transformed into DH5 competent cells (301, FIG. 3). A single colony was picked and allowed to grow in bacterial culture (302). The number of plasmid was expanded and extracted from the bacteria by mini-prep. The plasmid was further transformed into BL21DE3 or BL21DE3pLysS competent cells. Transformed bacteria were selected and grew in 2×100 ml LB medium. When the bacterial culture reached the optical density of 0.06, 200 μM of IPTG was added to 100 ml bacterial culture (303). Another 100 ml of bacterial culture without IPTG was used as negative control. The bacterial cultures were incubated at 30° C. with shaking. 500 μl of the bacterial cultures are saved and stored at −20° C. 3 h after the incubation and in the next morning after incubating overnight.

Bacterial cultures with and without IPTG induction were mixed together in a 500 ml centrifuge bottle. Bacterial cells were collected by centrifugation at 9000 rpm for 20 min at 4° C. (304). 500 μl of supernatant was saved as another negative control and the remaining supernatant was discarded. The bacterial cultures and negative controls collected in different points were run on a SDS-PAGE to resolve the protein (305). The gel was then stained with Coomassie Blue overnight. After de-staining the gel, the protein induction was confirmed by checking the size and comparing with the negative controls.

### Example 2b: Protein Purification for HCC Biomarker Set

The bacterial cell pellets were resuspended in 10 ml solubilization buffer by vortexing at room temperature. Keeping the resuspended cells in 50 ml centrifuge tube on ice, the cells were completely lysed by sonication at amplitude 70% 10 rounds of 30 s with interval of 30 s (401, FIG. 4). The lysed cells were centrifuged at 10,000 rpm for 1 h at 4° C. (402). Supernatants were transferred into dialysis tubing and submerged in 1 L unfiltered starting buffer for 4-6 h at 4° C. with constant stirring (403). Dialysis was continued with another 1 L starting buffer overnight. The supernatant was further filtered with 0.22 gm filter disc and syringe. To the AKTA machine equipped with 0.1M Nickel sulfate charged HiTrap chelating column (404), filtered samples were loaded (405). A program was set at the AKTA machine that the eluent is collected in fractions automatically (406). Proteins purified from different fractions were checked by SDS-PAGE analysis (407).

### Example 3a: Protein Coupling with BioPlex™ Beads

The purified proteins of the HCC biomarker set were coupled with BioPlex™ beads (Bio-Rad) (501) according to the manufacturer's manual. In brief, uncoupled beads were vortexed for 30 s and then sonicated for 15 s. 1,250,000 beads were collected in a reaction tube by centrifugation of 100 μl bead at maximum speed for 4 min. After washing with 100 μl bead wash buffer by centrifugation, the beads were resuspended in 80 μl bead activation buffer. To the beads 10 μl 50 mg/ml freshly prepared S-NHS and 10 μl 50 mg/ml freshly prepared EDAC were added, followed by 20 min incubation in dark at room temperature (FIG. 6). The beads were then washed twice with 150 μl PBS.

To the washed beads, 10 μg proteins were added and the total volume was topped up with PBS to 500 μl, and allowed to incubate for 2 h with shaking in dark. Supernatant was removed after centrifugation at maximum speed for 4 min. 250 μl blocking buffer was added to the beads and shook in dark for 30 min, followed by centrifugation at maximum speed for 4 min and removal of supernatant. The beads were briefly washed and then resuspended in the storage buffer for storage at 4° C. The numbers of the beads were counted with a hemocytometer.

### Example 3b: Validation of Protein-Bead Coupling

To a HTS 96 well plate, 50 μl of conjugated BioPlex™ beads (100 beads/μl) was added to react with HCC biomarker auto-antibodies followed by secondary antibodies (502). A serial dilution of the commercially available anti-bodies against the HCC biomarker set was prepared as 8,000, 4,000, 1,000, 250, 62.5, 15.625, 3.906, 0.977, 0.244 and 0.061 ng/ml. 50 μl of each dilution was added to each well. Two negative controls were performed by excluding the HCC auto-antibodies, and both the HCC auto-antibodies and secondary antibodies in the wells. The plate was then sealed with a foil and kept on a shaker for 30 min at 350 rpm, avoiding exposure to light.

After incubation, the beads were washed three times with 150 μl PBS. 50 μl of PE-conjugated secondary antibody (8,000 ng/ml) was added into each well except negative controls. The plate was sealed again and incubated in dark for 30 min with shaking. Excess antibodies were then washed away by PBS. The BioPlex™ machine was calibrated with the calibration kit and validation kit. After the HTS plate was loaded to the machine, signals from both the BioPlex™ beads and the PE conjugated at the secondary antibodies (503) were measured (schematic diagram is shown in FIG. 7). A calibration curve was generated by Logistic-SPL.

### Example 3c: Collection of Serum Samples and Measurement of Auto-Antibodies by Bioplex™ System

Whole-blood samples were clotted by standing at 37° C. for 1 h. Sera containing the auto-antibodies was collected at the supernatant after centrifugation at 1000 g room temperature for 10 min. The serum samples were diluted with PBS when necessary. To a HTS plate preloaded with Bioplex™ beads conjugated with a HCC biomarker set, the serum samples were loaded and incubated for 30 min with shaking (FIG. 13). Similar to the steps described in Example 3b, to the PBS washed beads, 50 μl of PE-conjugated secondary antibody (8000 ng/ml) was added, followed by shaking for another 30 min. After three rounds of washing, the plate was loaded to the BioPlex™ machine and the fluorescence signal was measured (504). The concentration of the auto-antibodies can then be calculated from the standard curves.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments are chosen and described in order to best explain the principles of the invention and its practical application, thereby enabling others skilled in the art to understand the invention for various embodiments and with various modifications that are suited to the particular use contemplated. It is intended that the scope of the invention be defined by the following claims and their equivalence.

### Example 4: Bioplex™ Measurement Explained

As mentioned previously herein, the Bioplex™ machine simultaneously measures two signals from the complex. One of the signals is a unique fluorescence signal given off by the Bioplex™ bead, which is used to identify the HCC biomarker to which it is attached, while the second signal is given off by the PE, which indicates the presence of HCC biomarker auto-antibodies. (FIG. 7) One having skill in the art and familiarity with the Bioplex™ machine would know that this machine can confirm that there are auto-antibodies for all 16 HCC biomarkers present in the sera because the machine analyzes the signals associated with each bead, one at a time as described below.

The Bioplex™ beads are supplied with various regions (e.g., Regions 1-100). Each HCC biomarker is conjugated to one specific region of the Bioplex™ beads. For example, Biomarker 1 will be conjugated to Bioplex™ Bead Region 1, Biomarker 2 will be conjugated to Bead Region 2 and so on. Each region will offer one distinct recognition signal, which is different from the signal from the PE conjugated secondary antibody. As set forth in the specification, all HCC biomarker conjugated beads are loaded into a single well and sample serum is applied to the same well. Serum auto-antibodies will bind to the corresponding HCC biomarkers conjugated on the beads. PE conjugated secondary antibody is then applied and will bind to the auto-antibodies. Although the Bioplex™ machine picks up all beads from the single well, only one bead at a time enters the signal detection area. The Bioplex™ machine first identifies the region of the bead and then the amount of PE signal on that particular bead. The region identified corresponds to which biomarker is analyzed and the PE signal will reflect the amount of auto-antibodies that are binding to the biomarker.

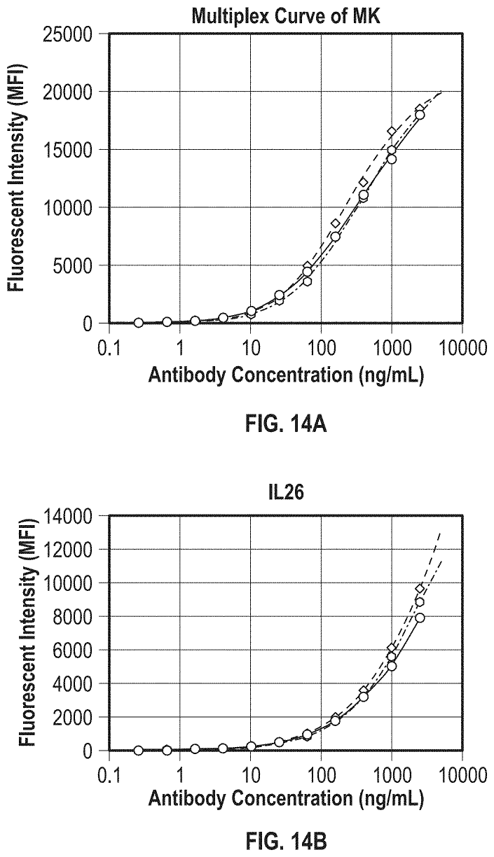

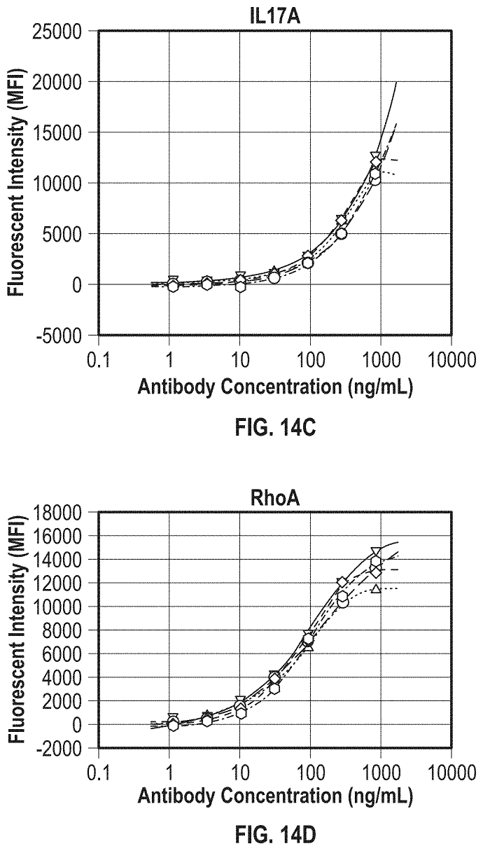

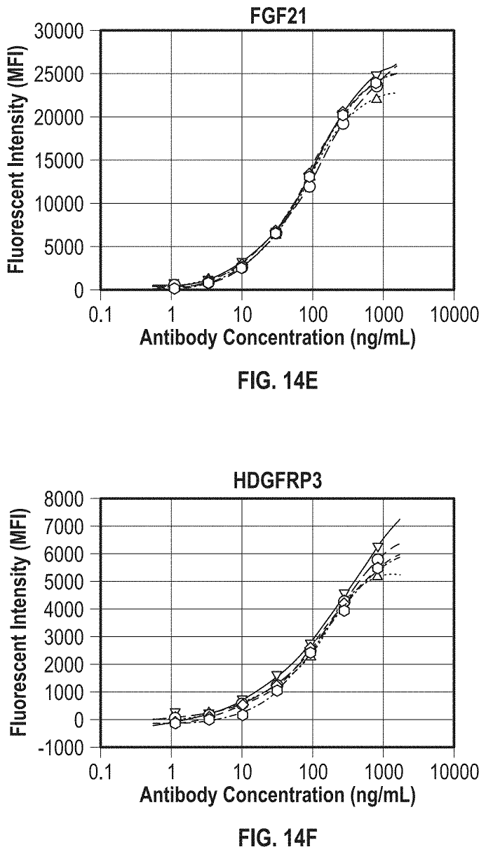

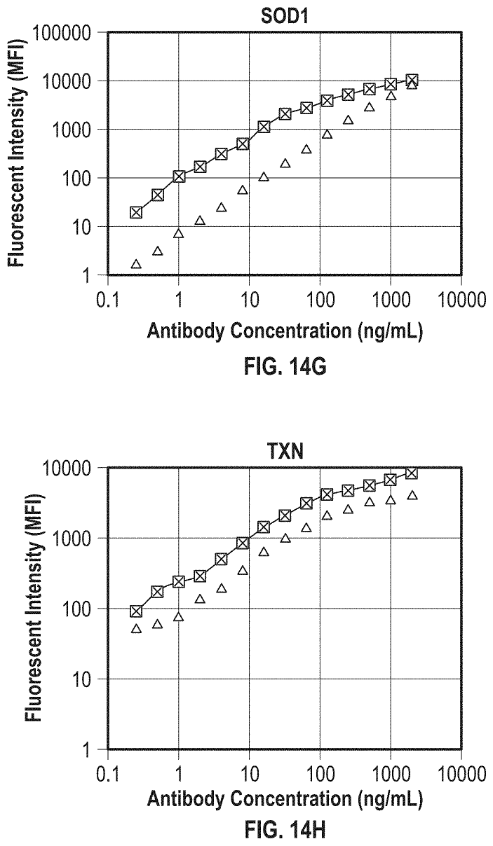

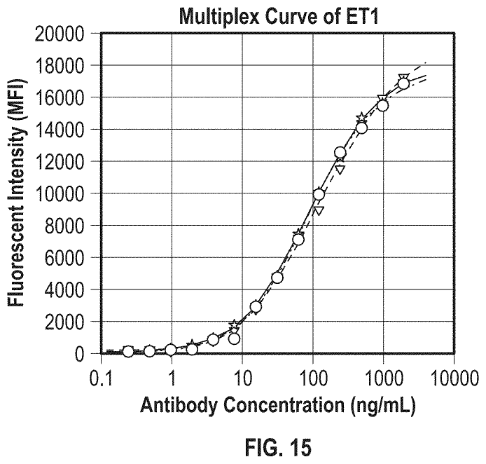

As set forth above, the concentration of the auto-antibodies can be calculated from the standard curves. The curve is fit with five-parameter logistics (5 PL) and an equation corresponding to the curve is automatically generated by the Bioplex™ machine. By substituting Y value (PE signal) into the equation, the applicants were able to calculate the X value (auto-antibody concentration). See FIGS. 14-15.

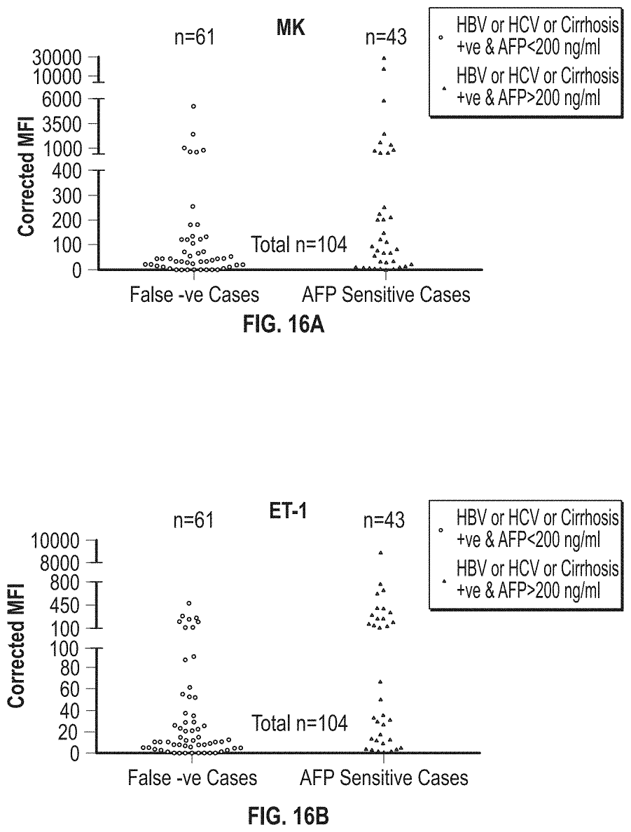

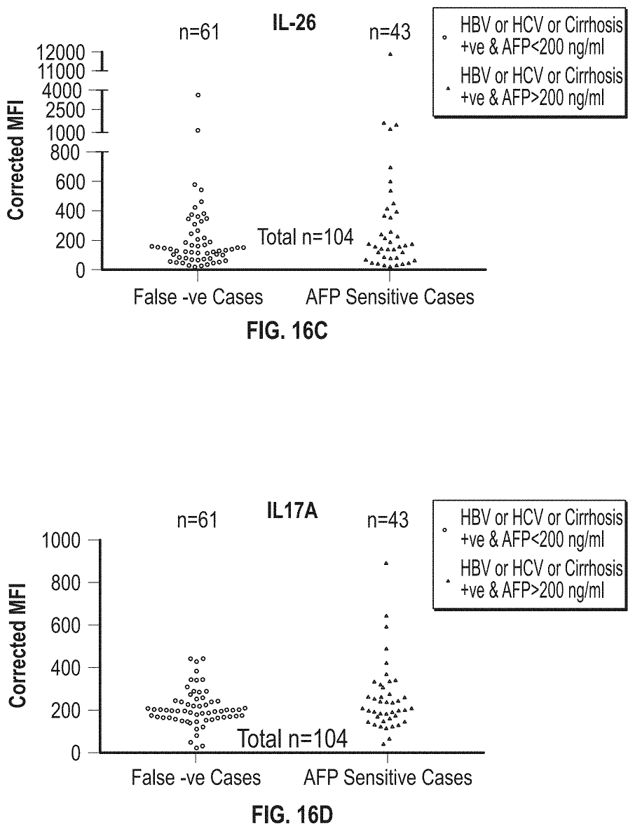

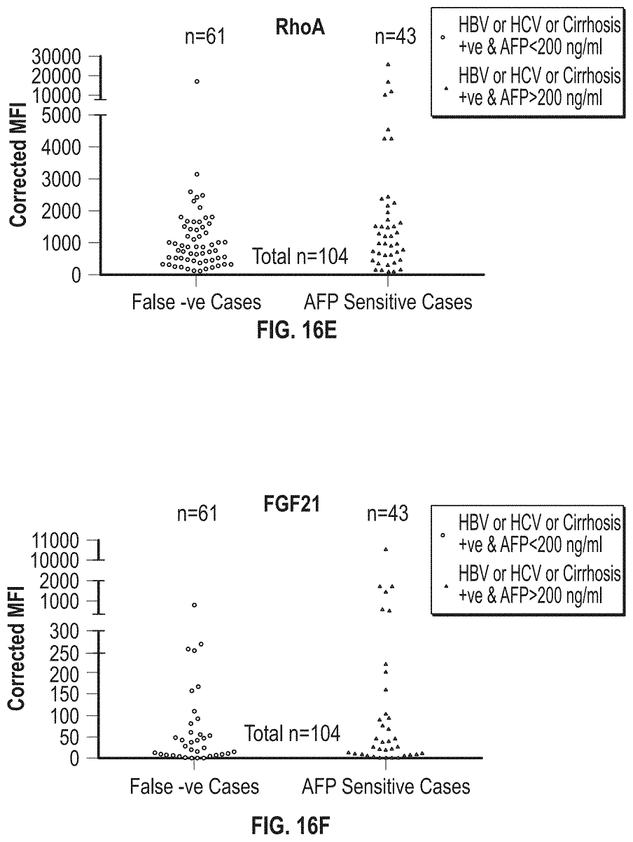

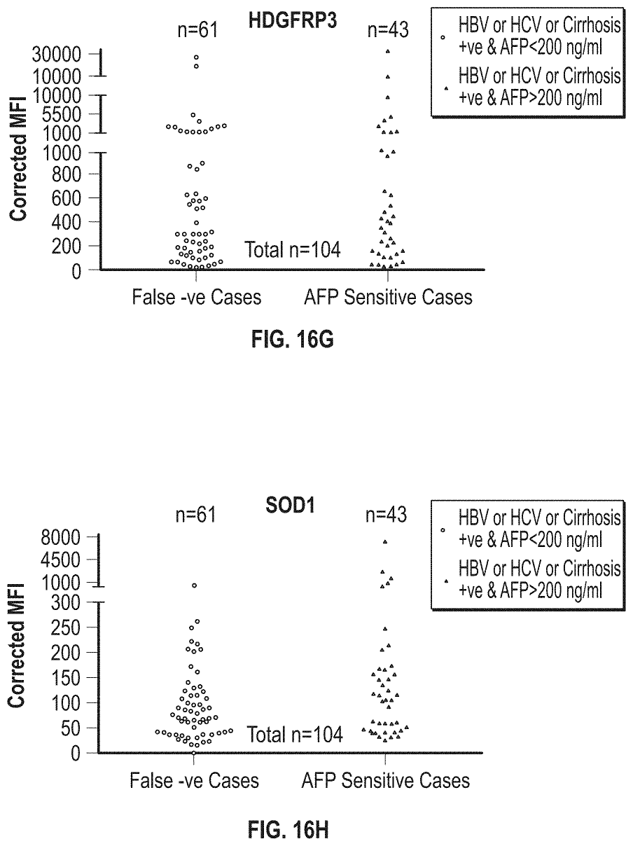

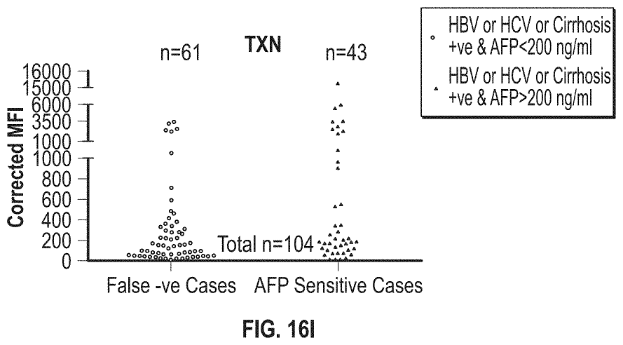

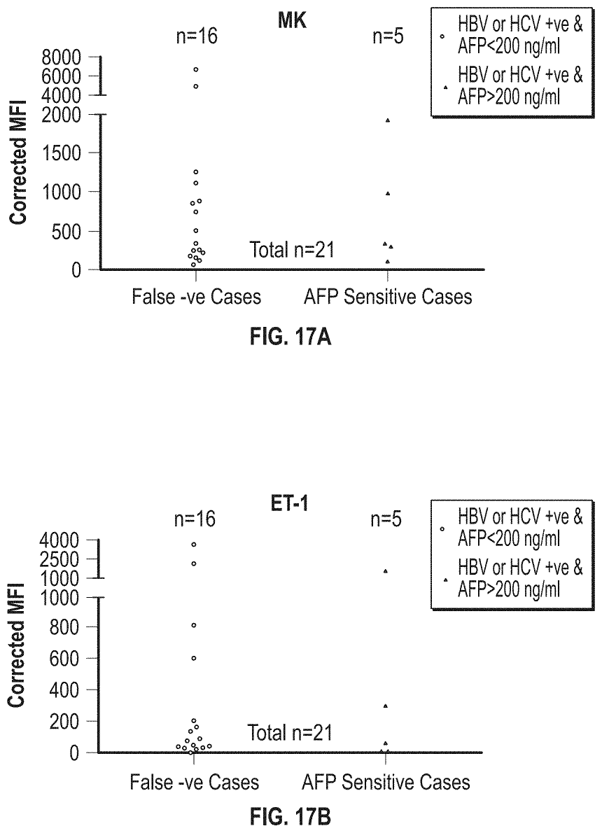

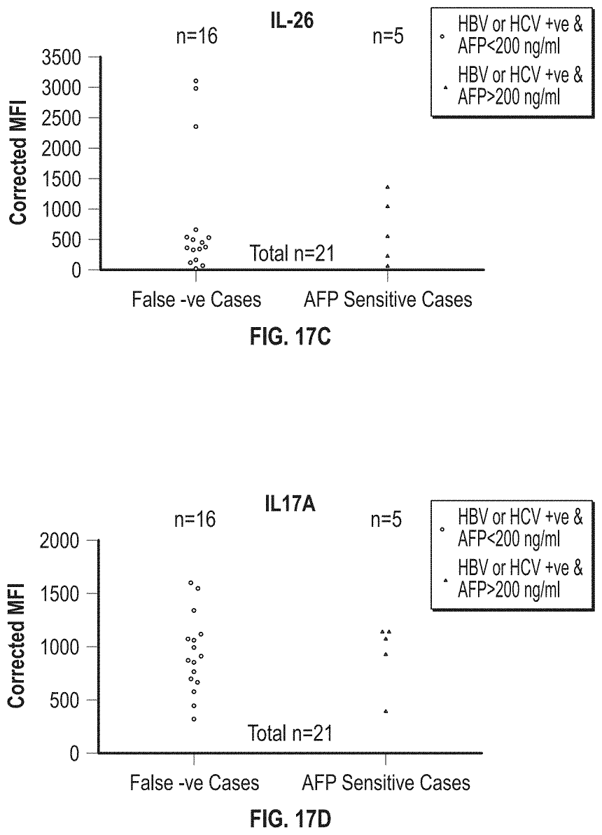

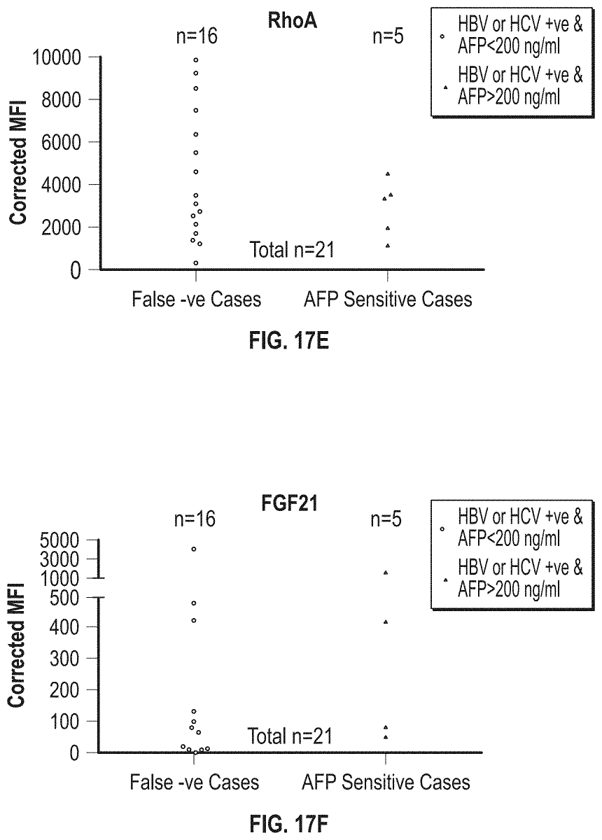

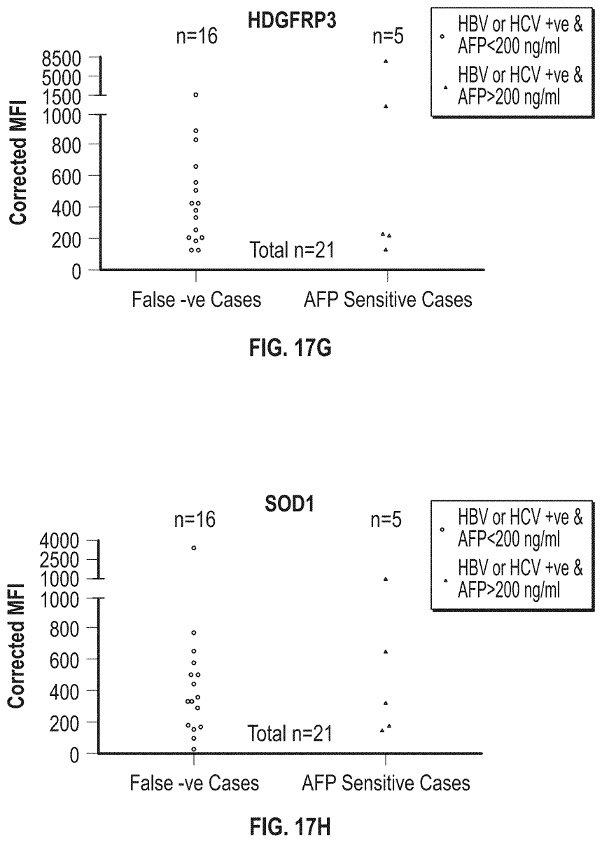

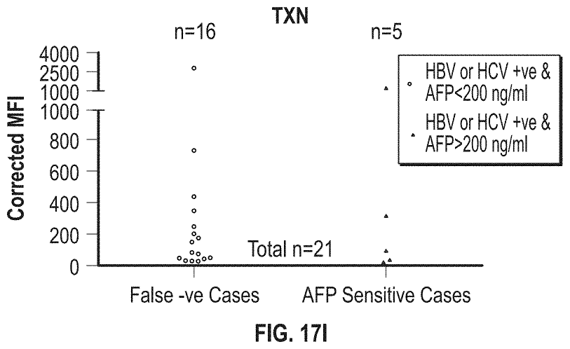

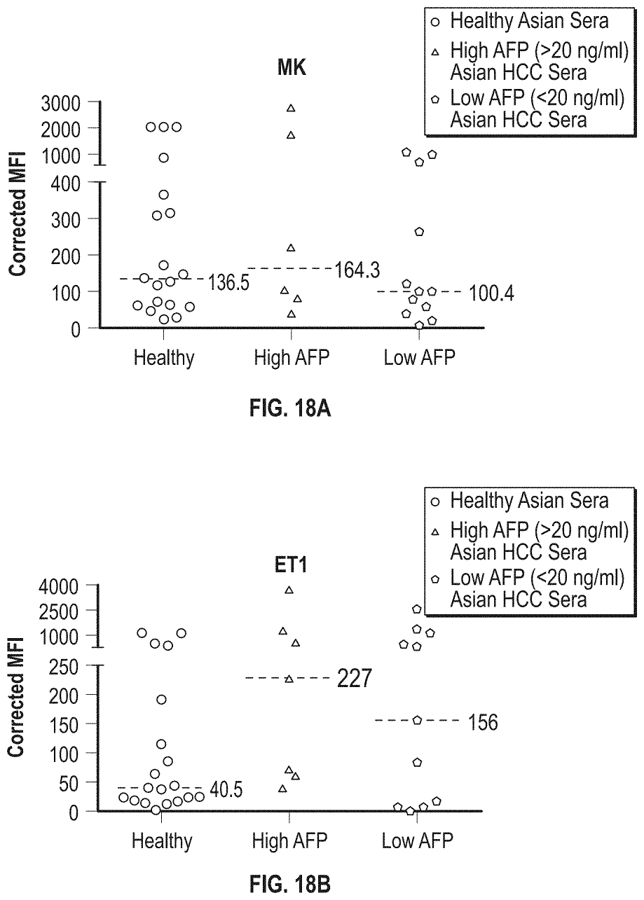

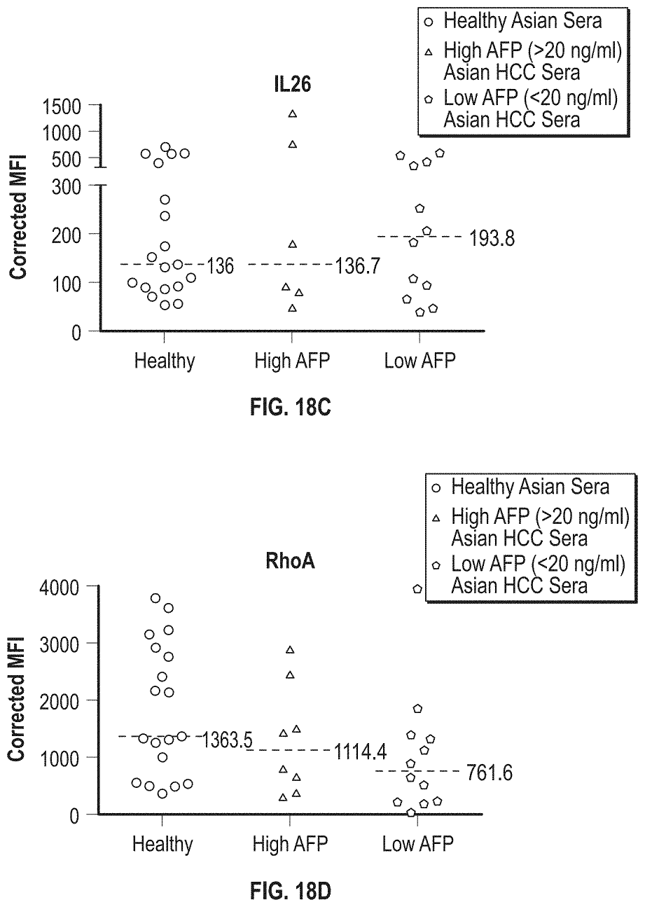

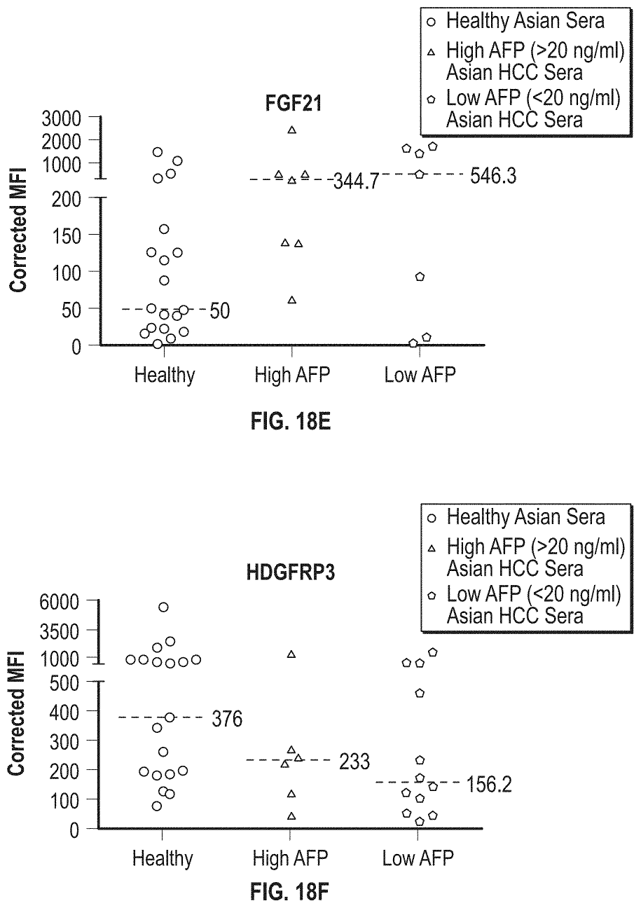

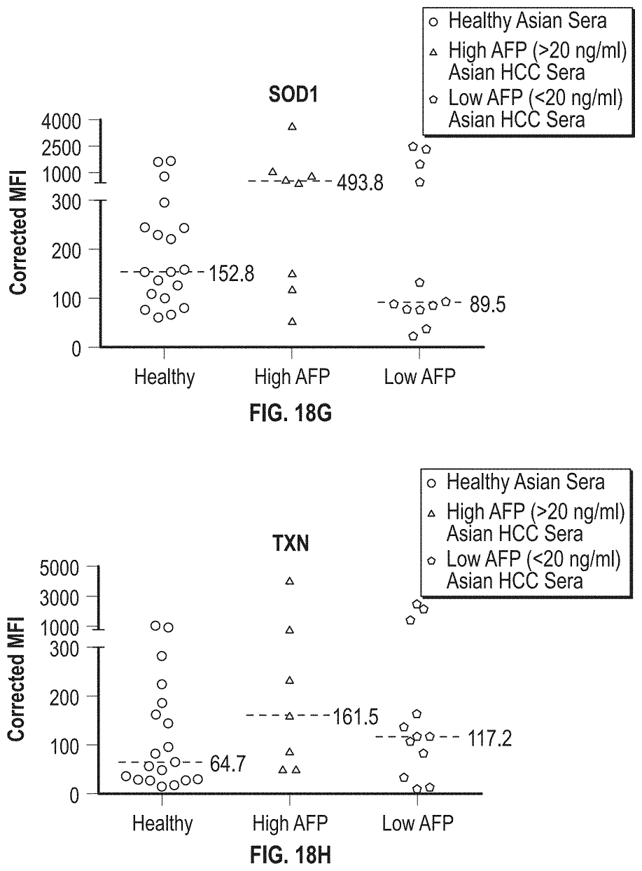

The presence of auto-antibodies in the sera was also validated by multiplex assay using Bioplex™ beads and the Bioplex™ machine as depicted below. Applicants tested healthy and liver cancer samples to generate auto-antibody signals of each biomarker. They used internal serum positive and negative controls to determine assay variation, which was kept below 20% according to FDA guidelines. The auto-antibody signal was subtracted from the naked bead signal to minimize non-specific signal binding to the bead surface (i.e., to ensure that the signal reflects the auto-antibody binding to its corresponding biomarker). The results show that auto-antibodies were present in all HCC serum detected, as depicted below where one dot represents one serum sample. See FIGS. 16A-16I; FIGS. 17A-17I; and FIGS. 18A-18H.

### Example 5: DCP

DCP (with 10 decarboxylation sites) conjugated beads were mixed with various healthy samples and HCC samples. The mixture was washed several times to remove non-specific auto-antibodies binding. Detection antibody was added to all samples and signal was detected by Bio-plex. The higher the signal, the higher the level of auto-antibodies against DCP was in the serum sample.

The test was performed in both Hong Kong and Canada sites. Results are very consistent that most of the tested HCC samples were having higher detected signal than healthy samples. A summary of detected signal is given in following table.

Average detected signal against DCP

| Ethnicity | Healthy | HCC |
|---|---|---|
| Asian | 102 MFI | 580 MFI |
| Caucasian | 94 MFI | 905 MFI |

MFI = Mean Fluorescent Intensity

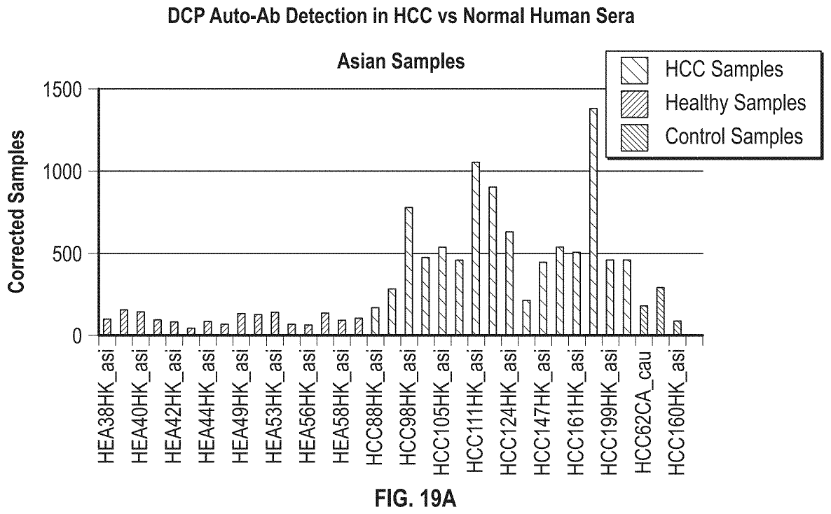

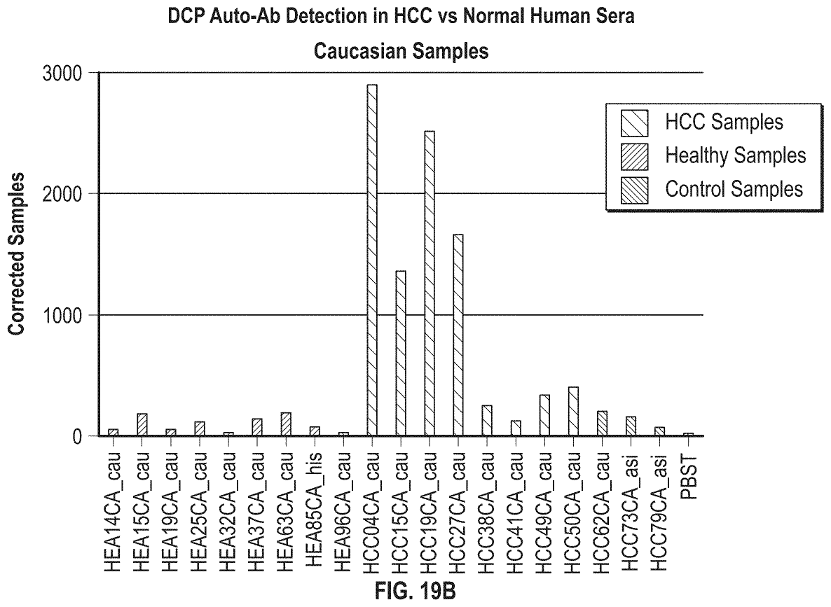

See also FIG. 19A and FIG. 19B which provide serum validation of DCP in healthy and HCC samples.

### Example 6: Auto-Antibody Measurement

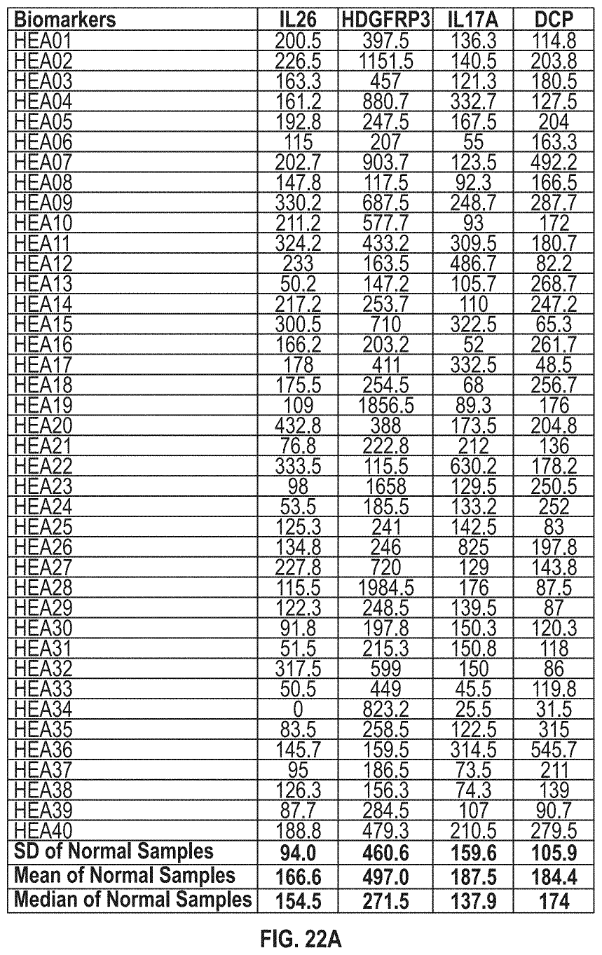

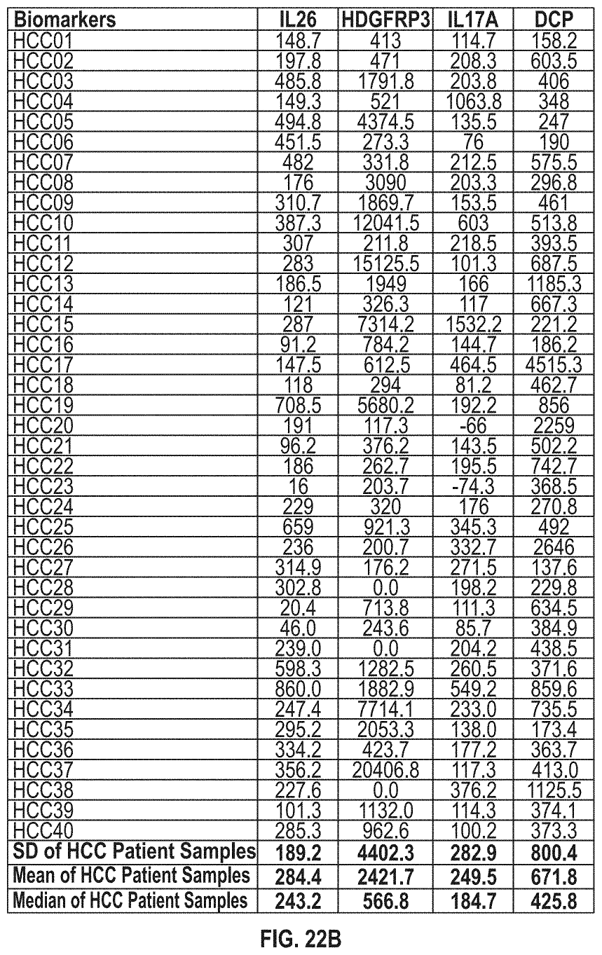

This example describes just one embodiment of the invention. Auto-antibodies against IL26, HDGF2, IL17A and DCP in 40 healthy person samples and 40 HCC patient samples were measured using the methods described herein—protein-bead conjugate and PE-conjugated secondary antibodies fed through the Bioplex™ machine. FIG. 22A shows the results of the measurement of auto-antibodies against IL26, HDGF2, IL17A and DCP in 40 healthy person samples. Each “HEAxx” is a sample from an individual known to be a healthy person. FIG. 22B provides the results for the HCC patient samples. Each “HCCxx” is a sample from an individual HCC patient. The numbers in the tables are the MFI (mean fluorescent intensity) measured from the PE-conjugated secondary antibodies showing the levels of auto-antibodies against the HCC biomarkers.

### INDUSTRIAL APPLICABILITY

The presently claimed method and kit comprising the 16 identified HCC biomarkers can not only be used to identify and quantify the presence of auto-antibodies in the patents' sera in order to detect and/or stage the liver cancer, but are also useful in drug development targeting these markers for specifically treating the liver cancer.

1. A method for measuring the presence of auto-antibodies against hepatocellular carcinoma (HCC) biomarkers in a subject suspected of having HCC, the method comprising:
a. obtaining a serum sample from the subject suspected of having HCC and measuring the serum for the presence of auto-antibodies against a set of HCC biomarkers, wherein the set of HCC biomarkers includes DCP and at least one other HCC biomarker selected from the group consisting of Bmi-1, VCC1, SUMO-4, RhoA, TXN, ET-1, UBE2C, HDGF2, FGF21, LECT2, SOD1, STMN4, Midkine, IL-17A, and IL26;
b. detecting the presence of the HCC biomarkers in the subject suspected of having HCC, the method comprising the steps of:
i. mixing the serum sample with a set of HCC biomarker conjugates to allow the auto-antibodies to the HCC biomarkers, if present in the serum sample, to bind to a set of HCC biomarker conjugates and washing away any unbound auto-antibodies;
wherein the set of HCC biomarker conjugates comprises each of the HCC biomarkers in the set of HCC biomarkers conjugated via an amide bond to a unique fluorescent microsphere bead,
wherein each unique fluorescent microsphere bead associated with a specific particular HCC biomarker in the set of HCC biomarkers has a different emission wavelength for each HCC biomarker,
wherein the HCC biomarker conjugates are capable of being bound by a specific auto-antibody against an HCC biomarker present in the subject's serum sample,
ii. adding to the mixture formed in step i. an anti-human secondary antibody conjugated with phycoerythrin (PE), which is capable of binding the auto-antibodies to the HCC biomarkers; and allowing the anti-human secondary antibody conjugated with PE to bind to specific auto-antibodies that are bound to HCC biomarker conjugates to form a fluorescent bead-biomarker-auto antibody-PE conjugated antibody cascade, and washing away an unbound anti-human secondary antibody; and
iii. measuring the mixture formed in step ii. for the presence of the fluorescent bead-biomarker-auto antibody-PE conjugated antibody cascade to determine whether the subject's serum contained auto-antibodies to the HCC biomarkers.

2. The method of claim 1 wherein the HCC biomarkers in the HCC biomarker conjugates were expressed from cDNA clones.

3. The method of claim 1 wherein the measuring the serum for the presence of auto-antibodies against the HCC biomarkers comprises measuring for the presence of auto-antibodies to DCP and measuring for the presence of auto-antibodies to Bmi-1, VCC1, SUMO-4, RhoA, TXN, ET-1, UBE2C, HDGF2, FGF21, LECT2, SOD1, STMN4, Midkine, IL-17A, and IL26.

4. The method of claim 1 wherein the measuring the serum for the presence of auto-antibodies against the HCC biomarkers comprises measuring for the presence of auto-antibodies to DCP and measuring for the presence of auto-antibodies to RhoA, TXN, HDGF2, SOD1, IL-17A, and IL26.

5. The method of claim 1 wherein the measuring the serum for the presence of auto-antibodies against the HCC biomarkers comprises measuring for the presence of auto-antibodies to DCP and measuring for the presence of auto-antibodies to at least one of RhoA, TXN, HDGF2, SOD1, IL-17A, and IL26.

6. The method of claim 1 wherein the measuring the serum for the presence of auto-antibodies against the HCC biomarkers comprises measuring for the presence of auto-antibodies to DCP and auto-antibodies to HDGF2, IL-17A, and IL26.

7. The method of claim 1 wherein the measuring the serum for the presence of auto-antibodies against the HCC biomarkers comprises measuring for the presence of auto-antibodies to DCP and auto-antibodies to at least one of HDGF2, IL-17A, and IL26.

8. The method of claim 1 wherein the measuring the serum for the presence of auto-antibodies against the HCC biomarkers comprises measuring for the presence of auto-antibodies to DCP and HDGF2.

9. The method of claim 1 wherein the measuring the serum for the presence of auto-antibodies against the HCC biomarkers comprises measuring for the presence of auto-antibodies to DCP and HDGF2 and auto-antibodies to at least one of RhoA, TXN, SOD1, IL-17A, and IL26.

10. The method of claim 1 wherein the measuring the serum for the presence of auto-antibodies against the HCC biomarkers comprises measuring for the presence of auto-antibodies to DCP and HDGF2 and auto-antibodies to at least one of IL-17A, and IL26.

11. The method of claim 1 wherein the unique fluorescent signal from the microsphere beads serves to identify which HCC biomarker in the set of HCC biomarkers is present and wherein the signal from the PE indicates the presence of the HCC biomarker conjugate.

12. The method of claim 11, wherein fluorescent intensity given by the PE-conjugated secondary antibodies in the fluorescent bead-biomarker-auto antibody-PE conjugated antibody cascade is measured to allow the detection and quantification of the HCC biomarker auto-antibodies.

13. The method of claim 1 wherein measuring the presence of auto-antibodies against hepatocellular carcinoma (HCC) biomarkers is performed on a plurality of subjects having HCC at different stages.

14. A kit for detecting auto-antibodies to a plurality of hepatocellular carcinoma (HCC) biomarkers in a patient's serum, the kit comprising:
a. a set of 16 hepatocellular carcinoma (HCC) biomarker conjugates comprising HCC biomarker proteins set forth in SEQ ID NOs: 1-16, wherein each protein set forth in SEQ ID NOs: 1-16 is coupled to a different fluorescent microsphere bead having a different emission wavelength; and
b. a PE-conjugated secondary antibody capable of binding to all of the HCC biomarker auto-antibodies wherein the HCC biomarker auto-antibodies are to the HCC biomarker proteins k3 mi 1 VCC1, SUMO-4, RhoA, TXN, ET-1, UBE2C, HDGF2, FGF21, LECT2, SOD1, STMN4, Midkine, IL-17A, IL26 and DCP as set forth in SEQ ID NOs: 1-16.

15. The kit of claim 14, wherein the kit is capable of detecting an auto-antibody to any one of the hepatocellular carcinoma (HCC) biomarker present in the patient's serum when the auto-antibody is present at an amount as low as about 0.15 ng/mL.

16. A kit for detecting auto-antibodies to a plurality of hepatocellular carcinoma (HCC) biomarkers in a patient's serum, the kit comprising:
a. a set of hepatocellular carcinoma (HCC) biomarker conjugates comprising HCC biomarker protein DCP and at least one other HCC biomarker protein selected from the group consisting of HDGF2, Bmi-1, VCC1, SUMO-4, RhoA, TXN, ET-1, UBE2C, FGF21, LECT2, SOD1, STMN4, Midkine, IL-17A, and IL26, wherein each HCC biomarker protein is coupled to a different fluorescent microsphere bead having a different emission wavelength; and
b. a PE-conjugated secondary antibody capable of binding to the HCC biomarker auto-antibodies, wherein the HCC biomarkers auto-antibodies are to the HCC biomarker proteins DCP, HDGF2, Bmi-1, VCC1, SUMO-4, RhoA, TXN, ET-1, UBE2C, FGF21, LECT2, SOD1, STMN4, Midkine, IL-17A, and IL26.

17. A kit for detecting auto-antibodies to a plurality of hepatocellular carcinoma (HCC) biomarkers in a patient's serum, the kit comprising:
a. a set of hepatocellular carcinoma (HCC) biomarker conjugates comprising HCC biomarker proteins HDGF2 and DCP wherein each HCC biomarker protein is coupled to a different fluorescent microsphere bead having a different emission wavelength; and
b. a PE-conjugated secondary antibody capable of binding to the HCC biomarker auto- antibodies, wherein the HCC biomarkers auto-antibodies are to the HCC biomarker proteins HDGF2 and DCP.

18. The kit of claim 17 wherein the set of set of hepatocellular carcinoma (HCC) biomarker conjugates comprises:
c. at least one additional HCC biomarker protein conjugate comprising HCC biomarker proteins selected from the group consisting of Bmi-1,B VCC1, SUMO-4, RhoA, TXN, ET-1, UBE2C, FGF21, LECT2, SOD1, STMN4, Midkine, IL-17A, and IL26; and

19. The kit of claim 18 wherein the kit further comprises:
d. a PE-conjugated secondary antibody capable of binding to the HCC biomarker auto-antibodies, wherein the HCC biomarker auto-antibodies are to the HCC biomarker proteins Bmi-1, VCC1, SUMO-4, RhoA, TXN, ET-1, UBE2C, FGF21, LECT2, SOD1, STMN4, Midkine, IL-17A, and IL26.

20. The kit of claim 18 wherein the kit comprises:
d. a PE-conjugated secondary antibody capable of binding to all of the HCC biomarker auto-antibodies, wherein the HCC biomarker auto-antibodies are to the HCC biomarker proteins DCP, HDGF2, Bmi-1, VCC1, SUMO-4, RhoA, TXN, ET-1, UBE2C, FGF21, LECT2, SOD1, STMN4, Midkine, IL-17A, and IL26.

21. The method of claim 2 wherein DCP protein is produced from bacteria that are unable to perform carboxylation of the DCP protein thereby expressing a DCP protein having all 10 sites decarboxylated.

22. The kit of claim 14 wherein DCP protein in the HCC biomarker conjugates was produced from bacteria that are unable to perform carboxylation of the DCP protein thereby expressing a DCP protein having all 10 sites decarboxylated.


SEQUENCE LISTING (16 sequences)

SEQ ID NO: 1 - protein, 326 aa, Homo sapiens
MHRTTRIKITELNPHLMCVLCGGYFIDATTIIECLHSFCKTCIVRYLETSKYCPICDVQVHKTRPLLNIRSDKTLQDIVYKLVPGLFKNEMKRRRDFYAAHPSADAANGSNEDRGEVADEDKRIITDDEIISLSIEFFDQNRLDRKVNKDKEKSKEEVNDKRYLRCPAAMTVMHLRKFLRSKMDIPNTFQIDVMYEEEPLKDYYTLMDIAYIYTWRRNGPLPLKYRVRPTCKRMKISHQRDGLTNAGELESDSGSDKANSPAGGIPSTSSCLPSPSTPVQSPHPQFPHISSTMNGTSNSPSGNHQSSFANRPRKSSVNGSSATSSG

SEQ ID NO: 2 - protein, 119 aa, Homo sapiens
MKVLISSLLLLLPLMLMSMVSSSLNPGVARGHRDRGQASRRWLQEGGQECECKDWFLRAPRRKFMTVSGLPKKQCPCDHFKGNVKKTRHQRHHRKPNKHSRACQQFLKQCQLRSFALPL

SEQ ID NO: 3 - protein, 95 aa, Homo sapiens
MANEKPTEEVKTENNNHINLKVAGQDGSVVQFKIKRQTPLSKLMKAYCEPRGLSVKQIRFRFGGQPISGTDKPAQLEMEDEDTIDVFQQPTGGVY

SEQ ID NO: 4 - protein, 193 aa, Homo sapiens
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 5 - protein, 105 aa, Homo sapiens
MVKQIESKTAFQEALDAAGDKLVVVDFSATWCGPCKMIKPFFHSLSEKYSNVIFLEVDVDDCQDVASECEVKCMPTFQFFKKGQKVGEFSGANKEKLEATINELV

SEQ ID NO: 6 - protein, 212 aa, Homo sapiens
MDYLLMIFSLLFVACQGAPETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGNPSRERYVTHNRAHW

SEQ ID NO: 7 - protein, 179 aa, Homo sapiens
MASQNRDPAATSVAAARKGAEPSGGAARGPVGKRLQQELMTLMMSGDKGISAFPESDNLFKWVGTIHGAAGTVYEDLRYKLSLEFPSGYPYNAPTVKFLTPCYHPNVDTQGNICLDILKEKWSALYDVRTILLSIQSLLGEPNIDSPLNTHAAELWKNPTAFKKYLQETYSKQVTSQEP

SEQ ID NO: 8 - protein, 203 aa, Homo sapiens
MARPRPREYKAGDLVFAKMKGYPHWPARIDELPEGAVKPPANKYPIFFFGTHETAFLGPKDLFPYKEYKDKFGKSNKRKGFNEGLWEIENNPGVKFTGYQAIQQQSSSETEGEGGNTADASSEEEGDRVEEDGKGKRKNEKAGSKRKKSYTSKKSSKQSRKSPGDEDDKDCKEEENKSSSEGGDAGNDTRNTTSDLQKTSEGT

SEQ ID NO: 9 - protein, 209 aa, Homo sapiens
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 10 - protein, 151 aa, Homo sapiens
MFSTKALLLAGLISTALAGPWANICAGKSSNEIRTCDRHGCGQYSAQRSQRPHQGVDVLCSAGSTVYAPFTGMIVGQEKPYQNKNAINNGVRISGRGFCVKMFYIKPIKYKGPIKKGEKLGTLLPLQKVYPGIQSHVHIENCDSSDPTAYL

SEQ ID NO: 11 - protein, 153 aa, Homo sapiens
MATKAVCVLKGDGPVQGIINFEQKESNGPVKVWGSIKGLTEGLHGFHVHEFGDNTAGCTSAGPHFNPLSRKHGGPKDEERHVGDLGNVTADKDGVADVSIEDVISLSGDHCIIGRTLVVHEKADDLGKGGNEESTKTGNAGSRLACGVIGIAQ

SEQ ID NO: 12 - protein, 189 aa, Homo sapiens
MTLAAYKEKMKELPLVSLFCSCFLADPLNKSSYKYEADTVDLNWCVISDMEVIELNKCTSGQSFEVILKPPSFDGVPEFNASLPRRRDPSLEEIQKKLEAAEERRKYQEAELLKHLAEKREHEREVIQKAIEENNNFIKMAKEKLAQKMESNKENREAHLAAMLERLQEKDKHAEEVRKNKELKEEASR

SEQ ID NO: 13 - protein, 143 aa, Homo sapiens
MQHRGFLLLTLLALLALTSAVAKKKDKVKKGGPGSECAEWAWGPCTPSSKDCGVGFREGTCGAQTQRIRCRVPCNWKKEFGADCKYKFENWGACDGGTGTKVRQGTLKKARYNAQCQETIRVTKPCTPKTKAKAKAKKGKGKD

SEQ ID NO: 14 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 15 - protein, 171 aa, Homo sapiens
MLVNFILRCGLLLVTLSLAIAKHKQSSFTKSCYPRGTLSQAVDALYIKAAWLKATIPEDRIKNIRLLKKKTKKQFMKNCQFQEQLLSFFMEDVFGQLQLQGCKKIRFVEDFHSLRQKLSHCISCASSAREMKSITRMKRIFYRIGNKGIYKAISELDILLSWIKKLLESSQ

SEQ ID NO: 16 - protein, 622 aa, Homo sapiens
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE